(19) United States
(12) Patent Application Publication
(10) Pub. No.: US 2024/0130653 A1
(43) Pub. Date: Apr. 25, 2024

(54) Method And System For Characterizing Human Urination And Data-Driven Management Of Urinary Health

(71) Applicant: Board of Trustees of Michigan State University, East Lansing, MI (US)

(72) Inventor: Subir K. BISWAS, Okemos, MI (US)

(21) Appl. No.: 18/487,094

(22) Filed: Oct. 14, 2023

Related U.S. Application Data
(60) Provisional application No. 63/419,029, filed on Oct. 25, 2022.

(51) Int. Cl. A61B 5/20; A61B 5/00

(52) U.S. Cl. CPC A61B 5/20; A61B 5/6891

(57) ABSTRACT

A urination monitoring system for a commode includes a housing coupled to the exterior of the commode, a vibration sensor disposed within the housing generating a vibration signal corresponding to vibration of an exterior of the commode, a signal shaping circuit coupled to the vibration sensor generating a processed vibration signal, a controller comprising a trained classifier coupled to the sensor receiving the processed signal and determining a urinary episode characteristic therefrom corresponding to at least one of a urinary frequency, urinary flow strength and duration, a number of episodes within a urination episode, a low strength and duration of sub-episodes within the episode and a display coupled to the controller generating an output the urinary episode characteristic.

### CROSS-REFERENCE TO RELATED APPLICATION

This application claims the benefit of U.S. Provisional Application No. 63/419,029, filed on Oct. 25, 2022. The entire disclosure of the above application is incorporated herein by reference.

### FIELD

The present disclosure relates to urinary health and, more specifically, to a method of characterizing human urination based on monitoring vibration signals.

### BACKGROUND

This section provides background information related to the present disclosure which is not necessarily prior art.

### SUMMARY

This section provides a general summary of the disclosure and is not a comprehensive disclosure of its full scope or all of its features.

The present disclosure provides a sensing arrangement that allows sensor attachment outside the commode as opposed to inside for typical sono-uroflowmetry. A dry solution allows a significantly more practical and durable solution, thus leading to potentially more successful implementation.

The present system provides a cyber physical system for data driven diagnostic, monitoring, and prediction of human urinary health. The vibration signal from a sensor is amplified, filtered, shaped, processed, and algorithmically analyzed for measuring: urination frequency, relative flow strengths and duration of each urine voiding episode, number of sub-episodes within each episode, the flow strength and duration of each such sub-episodes, and identifying the voiding subject for each episode.

In one aspect of the disclosure, a urination monitoring system for a commode includes a housing coupled to an exterior of the commode, a vibration sensor disposed within the housing generating a vibration signal corresponding to vibration of the exterior of the commode, a signal shaping circuit coupled to the vibration sensor generating a processed vibration signal, a controller comprising a trained classifier coupled to the sensor receiving the processed signal and determining a urinary episode characteristic therefrom corresponding to at least one of a urinary frequency, urinary flow strength and duration, a number of episodes within a urination episode, a low strength and duration of sub-episodes within the episode and a display coupled to the controller generating an output the urinary episode characteristic.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations and are not intended to limit the scope of the present disclosure.

FIG. 1A is a block diagrammatic view of the system according to the present disclosure.

FIG. 1B is a block diagrammatic view of the sensor module of FIG. 1A.

FIG. 1C is an example of a screen display for a device.

FIG. 1D is a screen display suited for providing a patient information.

FIG. 2A is a front view of a commode having the sensor module mounted thereon.

FIG. 2B is a cross-sectional view of a commode having a shield for mounting the sensor housing thereunder.

FIG. 2C is a perspective view of a bedside commode having the sensing system.

FIG. 2D is a cross-sectional view of the sensor of FIG. 2C.

FIG. 3 is a partially exploded view of the vibration sensor within the housing.

FIG. 4 is a block diagrammatic view of the signal shaping circuit.

FIG. 5 is a plot of a sample urination episode with many sub-episodes and post episodes proceedings.

FIG. 6 is a sample noise signal caused by ambient background vibration.

FIG. 7 is a flowchart of a method of operating the system.

FIG. 8A is an example of the amplifier output prior to spectral subtraction.

FIG. 8B is the output of the amplifier after De-noising.

FIG. 9A is a plurality of urination signatures without flushing events in the commode.

FIG. 9B is a plurality of urination signatures with flush events.

FIG. 10A is a plot of a duration distribution of urination episodes from a first subject.

FIG. 10B is a plot of urination distributions of urination episodes from a second subject.

FIG. 11 is a table illustrating a data set for urination and ambient events.

FIG. 12 is a table of different subjects on combined data.

FIG. 13 is a table of subject classification performance illustrating a f-score accuracy.

FIG. 14 is a table of classification performance across different subjects on combined data using a reduced neural network.

FIG. 15 is a classification performance with a reduced network for subject identification.

FIG. 16A are statistics for the number of sub episodes in each urination episode for subject one.

FIG. 16B are statistics for the number of sub episodes in each urination episode for subject two.

FIG. 17A is a sub episode duration distribution force for the first subject.

FIG. 17B is a sub episode duration distribution force for the second subject.

FIG. 18A is a sub episode energy representing the flow strength for a first subject.

FIG. 18B is a sub episode energy representing the flow strength for a second subject.

FIG. 19A is a voltage versus time plot of an on-water urination event.

FIG. 19B is a voltage versus time plot of an on-solid urination event.

FIG. 20A is a spectrogram of on-water urination.

FIG. 20B is a scalogram of on-water urination.

FIG. 21A is a spectrogram of on-solid urination.

FIG. 21B is a scalogram of on-solid urination.

FIG. 22A is an on-water time domain signal of a raw signal of an event.

FIG. 22B is the signal of FIG. 22A band pass filtered in the 19-21 Hz range.

FIG. 22C is the signal of FIG. 22A band pass filtered in the 12-15 Hz range.

FIG. 22D is the signal of FIG. 22A band pass filtered in the 0.5-3 Hz.

FIG. 23A is an on-water frequency domain signal of a raw signal of an event.

FIG. 23B is the signal of FIG. 23A band pass filtered in the 19-21 Hz range.

FIG. 23C is the signal of FIG. 23A band pass filtered in the 12-15 Hz range.

FIG. 23D is the signal of FIG. 23A band pass filtered in the 0.5-3 Hz.

FIG. 24A is an on-solid time domain signal of a raw signal of an event.

FIG. 24B is the signal of FIG. 24A band pass filtered in the 19-21 Hz range.

FIG. 24C is the signal of FIG. 24A band pass filtered in the 12-15 Hz range.

FIG. 24D is the signal of FIG. 24A band pass filtered in the 0.5-3 Hz.

FIG. 25A is an on-solid frequency domain signal of a raw signal of an event.

FIG. 25B is the signal of FIG. 24A band pass filtered in the 19-21 Hz range.

FIG. 25C is the signal of FIG. 24A band pass filtered in the 12-15 Hz range.

FIG. 25D is the signal of FIG. 24A band pass filtered in the 0.5-3 Hz.

Corresponding reference numerals indicate corresponding parts throughout the several views of the drawings.

### DETAILED DESCRIPTION

Example embodiments will now be described more fully with reference to the accompanying drawings.

A plurality of clinical applications may leverage the data obtained by the present system. The data may be developed for diagnostics, disease progress, monitoring effects of prescription, and overall personalized treatment plan by the Urological health providers. Optionally, intervention feedback can also be provided to the patients for modifying voiding behavior, when applicable. Although the technology is not restricted to any specific demography, the initial commercialization of the proposed device/system will be targeted mainly towards elderly people staying at home, nursing home, and other residential settings. Possible diseases and conditions that may be determined include but are not limited to enlarged prostate, overactive bladder, nocturia, polyuria, incontinence management via predictive alerts, overall hydration management, predicting and managing urinary tract infection, water-economic flush volume based on the urination amount, water leakage detection due to faulty flap.

Remote diagnostics and monitoring services can be packaged along with the monitoring device itself. Clinics, nursing homes, and hospitals will be the customer base.

Non-medical products may also be formed from the present teachings. An environmentally-friendly commode flushing system may be provided An amount of water used for a post-urination flush is decided based on the amount of urine discharge. The product can be sold as a standalone device, either integrated or retrofitted with the commode. The commode-attached device estimates the amount of urine discharge, and it controls the flush duration for the minimum required water dispensing, thus saving a significant amount of water wastage. Market size for this would be 100s of millions, covering many homes, offices, and public places such as airports, train stations etc.

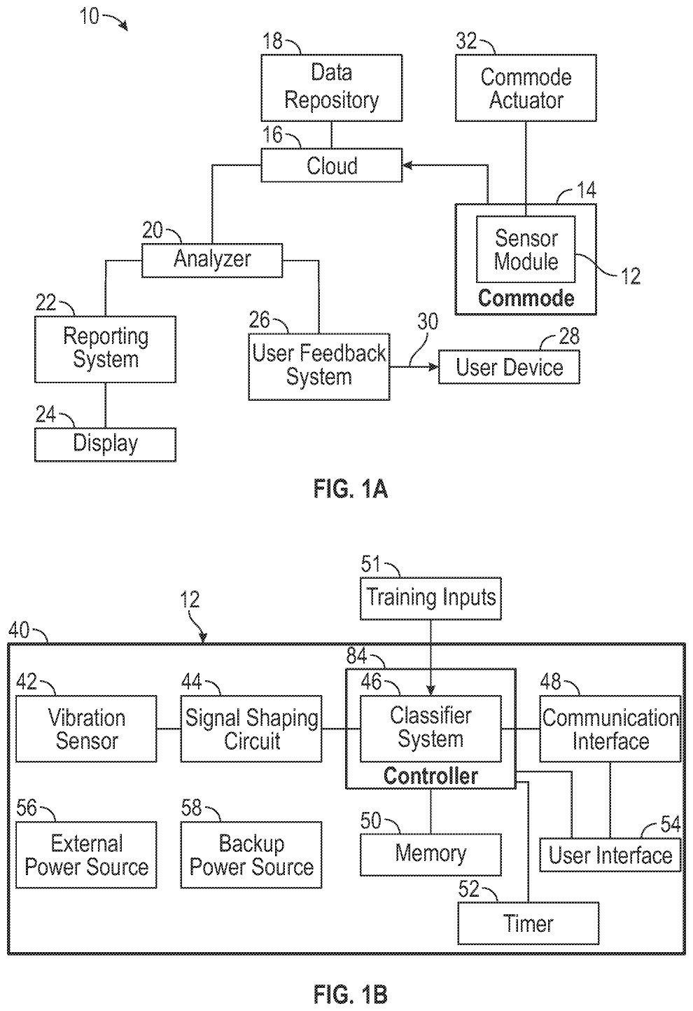

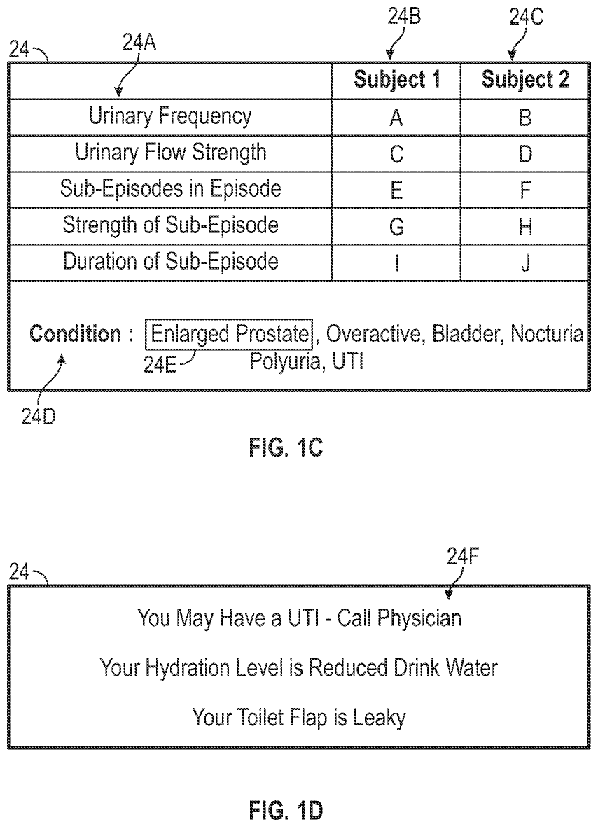

Referring now to FIG. 1A, a high-level block diagrammatic view of a monitoring system 10 is illustrated. The monitoring system 10 is a data driven diagnostic, monitoring and prediction system that, among other things, may be used to monitor human urinary health. A sensor module 12 is coupled to a commode 14. The commode 14 may be a toilet or other urinary receptacles. In the present example, the commode 14 is formed from porcelain having an interior with water therein and an exterior. The commode wall 14A divides the interior and exterior. Vibrations of the exterior wall 14A of the commode are caused by moving water due to urine contacting the water. The vibrations are detected by the sensor module 12. In the present example, the sensor module 12 is a match-box size sensor that is coupled to the commode 14.

The sensor module 12 may generate data or signal that contain data that communicates with the cloud 16. The cloud 16 represents the internet or another type of network. The cloud 16 may include a data repository 18 for storing historical data regarding the sensed conditions at the sensor module 12. The cloud 16 may include or be in communication with an analyzer 20 that is used to analyze the data provided from the sensor module 12. The analyzer 20 may be included within the cloud 16 or may be located at another location. The analyzer 20 may provide hydration, diagnostics and the predictive analysis based upon the data from the sensor module 12. The analyzer 20 and the sensor module 12 may be referred to collectively as a controller because various analysis and calculations may take place at either device, or at least partially at either device and communicated to the other device. The analyzer 20 may also provide such analysis based upon the data within the data repository 18. The analyzer 20 is coupled to a reporting system 22. The reporting system 22 may be used to generate reports on the various monitored conditions. A display 24 may display various monitored conditions associated with the analyzer 20. The display 24 may display one or more of the graphs set forth below or simplified versions with just the resulting numbers associated with the urination events and/or subevents. A condition or both a condition and data may also be displayed.

A user feedback system 26 is coupled to the analyzer 20. The user feedback system 26 may generate feedback to a user device 28 based upon the monitored conditions. For example, the user device 28 may comprise a handheld device such as a computer or mobile phone that has an application that generates a notification. Other types of devices may include a smart cup that is activated to inform the user that the amount of hydration is too low. Communication with the user device 28 may take place through a network 30. However, communication through the cloud 16 is also possible.

A commode actuator 32 may be coupled to the commode 14. The commode actuator 32 may be a flush valve that allows variable opening times to control the amount of water used to flush the commode 14. As described more later, the amount of flush water can be adjusted based on the amount of urine to conserve water.

Referring now to FIG. 1B, the sensor module 12 is illustrated in further detail. The sensor module 12 may be enclosed within a housing 40. The housing 40 includes a vibration sensor 42. The vibration sensor 42 may be a piezoelectric sensor that captures the minute vibration of the commode 14 when the flow of urine falls on the water within the commode. The vibration sensor 42 generates an electrical signal that is shaped by the signal shaping circuit 44. The signal shaping circuit 44 amplifies and shapes the signal as will be described in more detail below. Ultimately, the signal shaping circuit 44 is provided to a controller 84. The controller 84 is microprocessor-based. Some functions of the controller 84 may be performed at the analyzer 20. One or more microprocessors may be used within the controller 84. The controller 84 has a classifier system 46 used for classifying the process signals from the signal shaping circuit 44. Details of the classifier system 46 are provided in more detail below. The classifier system 46 algorithmically analyzes the processed electrical signals and may measure various conditions such as urination frequency, the relative flow strengths and duration of each urination episode, the number of sub-episodes within each urination episode, the flow strength and duration of each sub-episode and identifying the urination subject for each episode. The present disclosure expands traditional uroflowmetry that typically only measures the flow strength of the urination process for diagnostics of the bladder and prostate of the subject. The signals are ultimately communicated to the analyzer 20 of FIG. 1A. The classification provided by the classifier system 46 is communicated in a classification signal to the analyzer 20 of FIG. 1A. Ultimately, various types of disorders including an enlarged prostate, overactive bladder, nocturia, polyuria, incontinence management via predictive alerts, overall hydration management, predicting and managing urinary tract infections and water economic flush volume based upon the urination amount may be provided. Ultimately, the disorders can diagnose and monitor disease progression and the effects of treatments and the predictions of various time scales. Hydration management may take place by communicating with the user device 28 described above. The user of smart bottle as the user device 28 of FIG. 1A may monitor an alert to the subject to provide further hydration or indicate that the hydration level is acceptable. This is particularly useful for elderly individuals that live alone or in nursing home settings. Water economic automated commode flushing may also take place. As illustrated in FIG. 1A, a commode actuator 32 may be coupled to the sensor module 12. Based upon the duration, flow and other detected properties of a urination episode, the amount of water used for flushing the commode may be adjusted to conserve water.

Ultimately, the controller 84 is in communication with a communication interface 48. The communication interface 48 may provide wired or wireless communication through the cloud 16 or other networks 30 to the analyzer 20. The process and classification signals from the classifier system 46. The controller 84 may also be associated with a memory 50 that is used for storing various data used in the processing of the classifier system. For example, training inputs 51 may be used in the classifier system. Ultimately, a neural network may be trained. The memory 50 may store intermediate data or the like used by the controller 84.

A timer 52 is also associated with the controller 84. The timer 52 is used for timing various events and time periods associated with the classifier system 46 and the controller 84. A user interface 54, such as buttons or a touch screen, may also be associated with the controller 84. The user interface 54 may be used for initiating the operation, entering various data and overall control of the controller 84. The user interface 54 may also be associated with the communication interface. That is, external devices may be in communication wirelessly or through a wire to provide data to and from the controller 84. The housing 40 may also have an external power source 56 associated therewith. The external power source 56 may be an AC or DC power source used to provide power to the components within the housing 40. The external power source 56 may be provided directly from or transformed from the power of a building or home. A backup power source 58, such as a battery, may be used when the external power source 56 has been disconnected. The backup power source 58 may be charged by the external power source 56 to be used when the external power source fails.

Referring now to FIG. 1C, an example of a screen display 24 is provided. The display 24, in this example, may be suitable for a clinical or physician environment in which one or more subjects may be analyzed. In this example, a data area 24A is provided with different types of data such as the urinary features, the urinary flow strength, sub-episodes in the urinary episode, strength of sub episodes and the duration of the sub-episodes. The data for subject 1 is provided in a first data area 24B and the data for subject 2 is provided in a second data area 24C. Although no numbers are provided in the data areas 24B, 24C, the letters A-J represent potential data.

A condition area 24D may also be provided on the display. The condition area 24D may be, in addition to the areas 24A-24C or instead of data areas 24A-24C. In this example, a number of conditions may be listed and highlighted by a selected area 24E. In this case, the highlighted area 24E corresponds to “enlarged prostate”. However, other types of conditions, such as overactive bladder, nocturia polyuria and urinary tract infection (UTI) are provided. One or more conditions may be selected by the condition selector 24E. Of course, one single condition may be displayed.

Referring now to FIG. 1D, the screen display 24 is illustrated with condition messages 24F. In this example, three examples of conditions are provided. However, only one or more than one conditions may be provided on the display 24. Examples of conditions provided in this example are, “you may have a UTI-Call a physician”, Your hydration level is low-drink water” and “your toilet flap is leaky.” In a practical example, one message may be displayed.

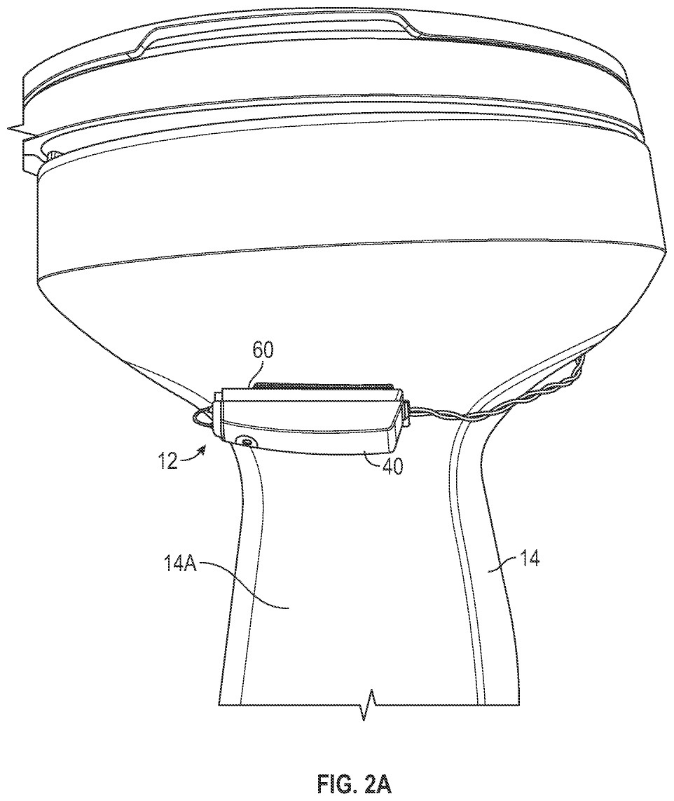

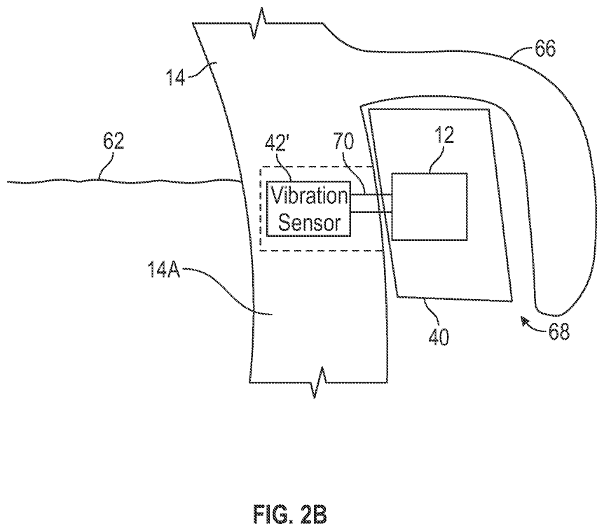

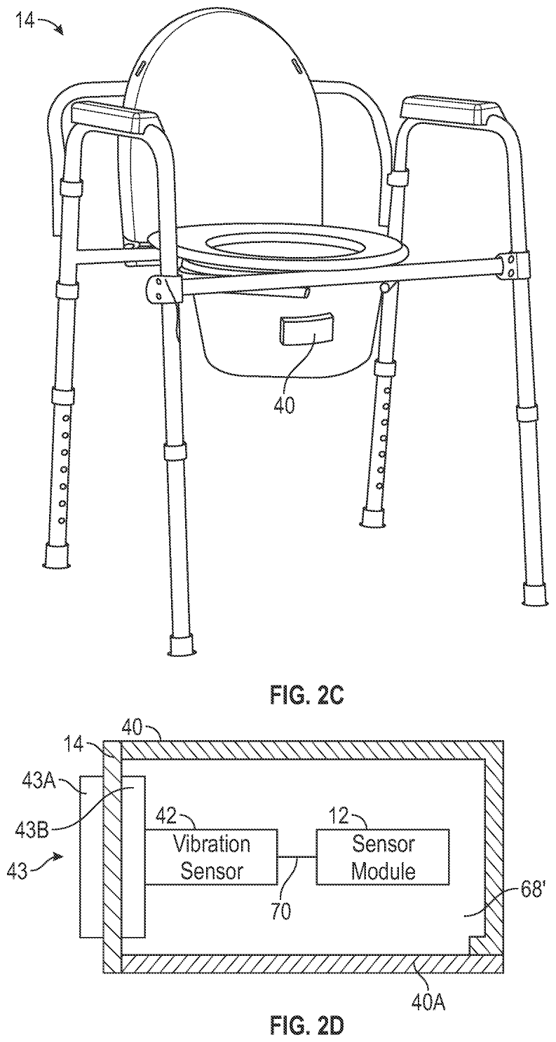

Referring now to FIG. 2A, a commode 14 is illustrated having the sensor module 12 coupled thereto. The housing 40 of the sensor module 12 is fastened to the commode using a fastener 60 such as hook and loop fastener. The fastener 60 should be such that the urinary episode and the vibrations caused thereby are transmitted through the commode to the housing 40 and ultimately to the vibration sensor 42 disposed therein. The housing 40 is coupled to the commode 14, in this example, at or below a water line 62. During testing, it was found that the turbulence created by the intersection of the urine and the commode water creates a vibration on the commode 14 which is transmitted through the wall 14A of the commode 14 and may be detected by the sensor module 12. As illustrated in FIG. 2A, the housing is an external device coupled to the commode 14. Referring now to FIG. 2B, in another example, the commode 14 may have a shield 66 formed therein. The shield 66 may be integrally formed with the material of the commode 14. The shield 66 may be sized to form a volume 68 between the shield 66 and the commode 14. The volume 68 is sized to receive the housing 40 and the sensor module 12 therein. In this example, the sensor module 12 may include the vibration sensor. However, a vibration sensor 42′ may be embedded within the wall 14A of the commode 14. The vibration sensor 42′ may be coupled through wires 70 to the sensor module 12. However, the vibration sensor 42′ may also be magnetically coupled to the sensor module 12 in various environments such as an institutional environment. Not all commodes may require monitoring. However, when monitoring is desired, the housing 40 with the sensor module 12 therein may be moved to the desired commode 14. When the commode 14 includes the shield 66, the visual aesthetics of the system are improved.

Referring now to FIGS. 2C and 2D, in another example, the commode 14 may be a bedside or portable commode. The commode 14 may be formed of metal, plastic or another type of composite material. In this example, the housing 40′ may be molded into or may be a stand-alone device. The volume 68′ delineated by the housing 40′ is sized to receive the sensor module 12 and the vibration sensor 42 therein. A door 40A is coupled to the housing 40 to enclose the housing. 40′. In this example, the sensor module 12 may include the vibration sensor 42. However, the vibration sensor 42 may be embedded or removably attached to the wall 14A of the commode 14 in various ways including but not limited to adhesive or fasteners. As illustrated, a magnetic coupler 43 has a first portion 43A and a second portion 43B that are magnetic and coupled through a composite material of the commode. The portions may have one of 43A or 43B formed from a magnet and the other formed of a magnetic material such as steel. If the commode 14 is formed of steel, only a magnetic portion 43B is required to hold the sensor 42 to the commode 14. The vibration sensor 42 may be coupled through wires 70 to the sensor module 12.

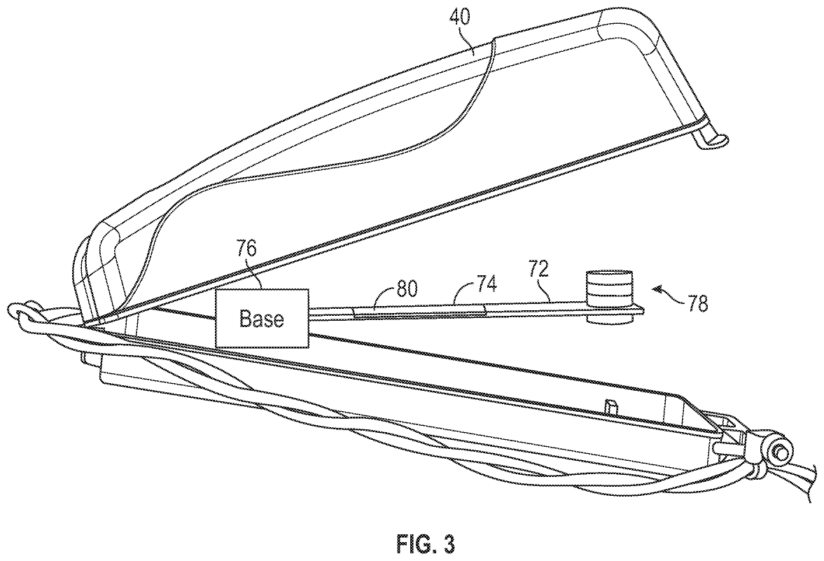

Referring now to FIG. 3, a piezoelectric sensor 72 is illustrated as the vibration sensor 42. The piezoelectric sensor 72 includes a cantilever 74 that is coupled to a fixed base 76 within the housing 40. The base 76 has a fixed position within the sensor housing 40. The cantilever 74 has a ballast 78 on the end thereof. The ballast 78 is on the opposite end of the cantilever 74 as the vibration sensor 42. Although a piezoelectric sensor 72 is used as the vibration sensor 42, other types of sensors may act as a vibration sensor. The cantilever 74 and the ballast 78 improves the vibration characteristics of the system. The end of the cantilever 74 with the ballast 78 vibrates and allows the vibration to persist for longer due to the momentum of the ballast 78. The vibration of the piezoelectric cantilever 74 creates a voltage signal in the piezoelectric layer 80 which is subsequently processed and analyzed as described above. This arrangement provides an ultra-sensitive vibration sensor that can detect even a few drops of fluid in the commode. As will be described, noise management due to the environmental conditions such as human activities within a bathroom are filtered.

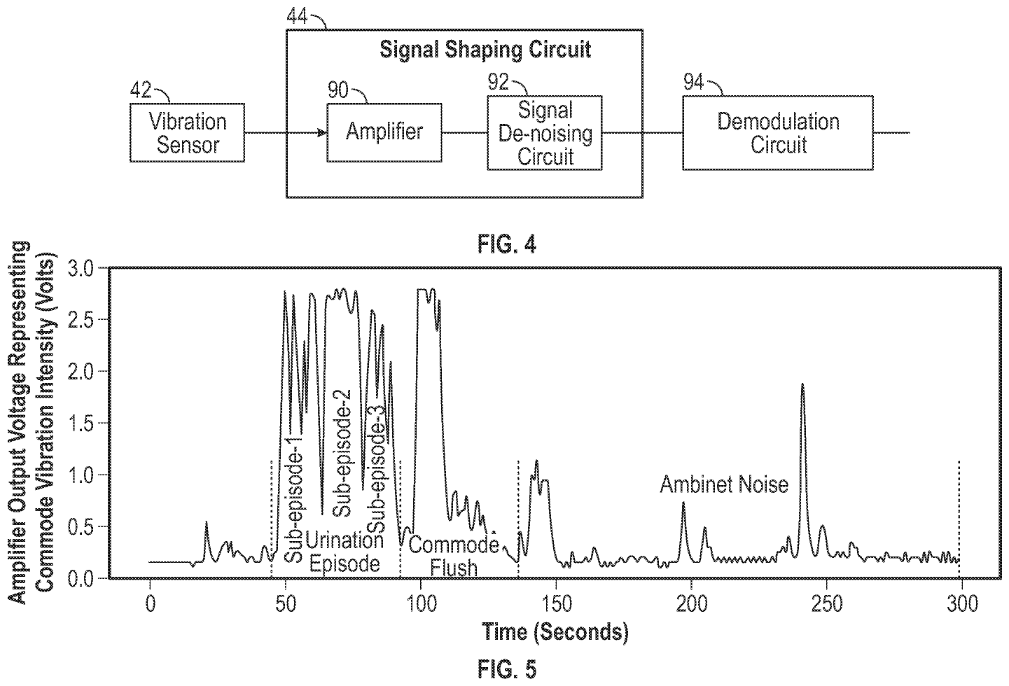

Referring now to FIG. 4, the vibration sensor 42 is coupled to the signal shaping circuit 44 as described in FIG. 1B. In this example, the electrical signal provided by the vibration sensor is communicated to an amplifier 90. The amplifier 90 may be powered by the external power source 56 or the backup power source 58 disposed within the housing 40. This allows continuous operation of the system even during a temporary power outage.

The amplifier 90 is coupled to a signal de-noising circuit 92. The signal de-noising circuit 82 may filter the amplified signal from the amplifier 90 to reduce the amount of external noise that is provided to the classifier system 46 of FIG. 1B. The signal de-noising circuit 82 may include various types of filtering including bandpass filtering and the like. Both the amplifier 90 and the signal de-noising circuit 92 may be implemented in discrete circuitry or custom application specific integrated circuits (ASIC). The signal de-noising circuit 92 may also be implemented in digital signal processing.

A demodulation circuit 94 is in communication with to the de-noising circuit 92 The demodulation circuit 94 is used to demodulate the denoised signal to form a denoised and demodulated signal. The denoised and demodulated signal is used to obtain the urinary signatures for the various subjects.

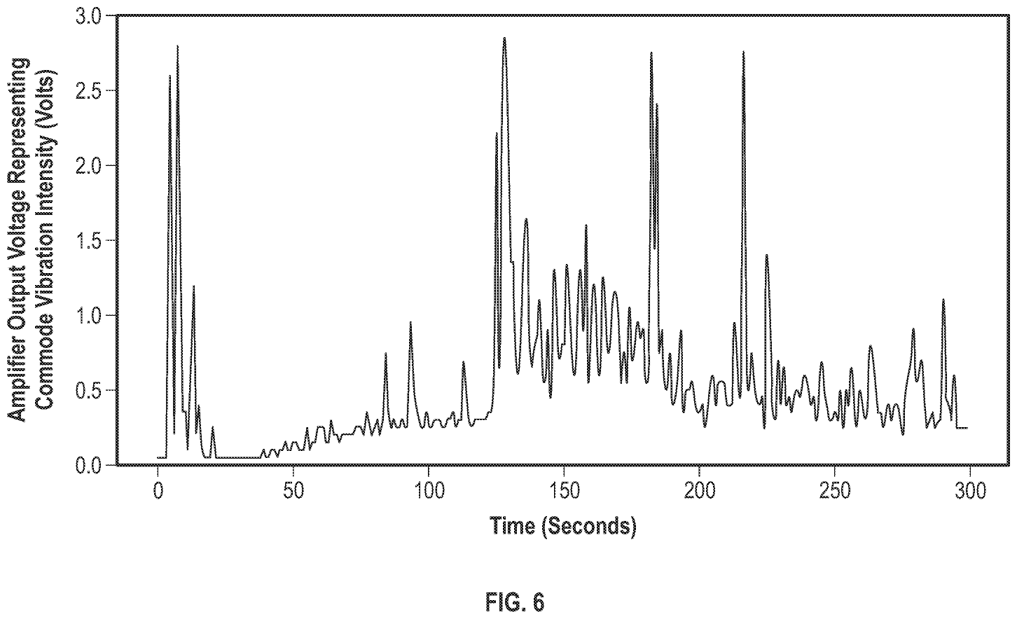

Referring now to FIGS. 5 and 6, the outputs of the amplifier 90 and any de-noising in the de-noising circuit 82 are captured by sampling. In this example, a 1 Hz sampling rate was used. The graph in FIG. 5 shows the voltage representing commode vibration signature for a valid urination episode, followed by that of the commode flush and ambient noise. FIG. 5 shows that the episode, lasting for about 40 seconds, has three main bladder emptying sub-episodes and their relative amplitudes representing the force of urine falling on the commode water. Additional information such as the tentativeness of the flow for each sub-episode is also available within the signal. The graph then shows vibration signal of a commode flush at the end of which there is some ambient noise lasting for almost 150 seconds.

FIG. 6 shows the commode vibration signature during which no urination was taking place. As can be observed that ambient noise vibrations with sufficiently high amplitudes can be present in the environment in the absence of urination. Such ambient vibrations are found to be caused by many factors including human walking, showering, even house vibrations due to wind etc.

It can be also observed that the nature and shape of such noise are very different from typical vibration signatures for urination. This was generally observed to be true for different times, different bathroom commodes, and different urinating subjects. As shown later in the document, the unique vibration signatures caused by urination can be successfully used not only to distinguish them from background noise, but person-specific signatures can be used for identifying subjects when they share a commode at different times.

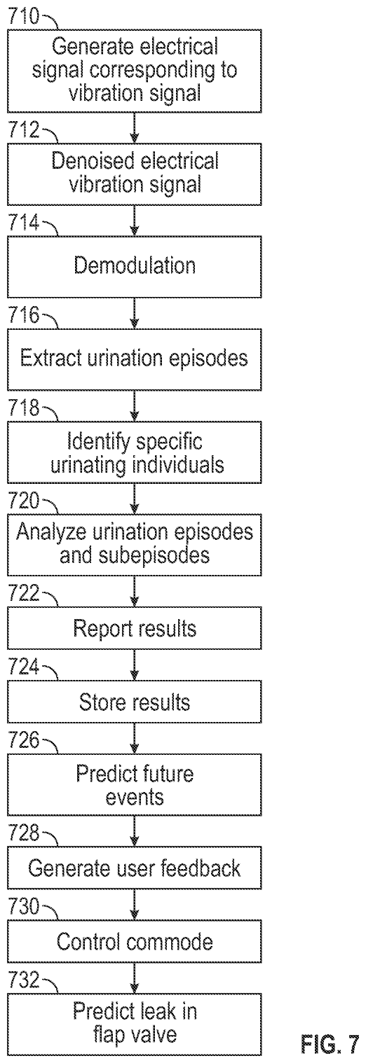

Referring now to FIG. 7, a high-level method of processing the signals is set forth. The analyzer 20 or the controller 84 or combinations of both may be used to perform the functions. In step 710, the raw electrical signal from the piezoelectric vibration sensor is generated in step 710. In step 712, the signal is de-noised. That is, the electrical vibration signal is de-noised in step 712. In step 714, the de-noised signal is demodulated to extract the urination signatures for urination. In step 716 urination episodes are extracted from the de-noised and demodulated electrical vibration signal. The urination episodes are determined based upon various classifications such as within a neural network. In step 718, urinating individuals may be identified. In step 720, the urination episodes and sub-episodes are analyzed. Ultimately, step 722 reports the results. In step 724, the results are stored for historical purposes. Based upon the current data and the historical data, step 726 may be used to predict future events. In addition, step 728 may generate user feedback to allow an indication of hydration of a user. In step 728, the system may also control a commode to flush using a predetermined amount of flushing fluids, such as water. In step 730, the system may also be used to predict a leak in a flap valve of a commode. That is, the trained classifier may be trained to monitor the vibration due to a leak in the flap valve and generate a flap leak signal corresponding thereto. This prevents an efficient use of water within the commode. The classifier system 46 may be trained to determine a leak in the flap valve.

The de-noising step 712 is set forth in further detail. The de-noising step 712 may be performed by spectral subtraction.

The raw signal out of the amplifier is de-noised so that the artifacts generated from human- and ambience-generated noise can be removed. For this purpose, spectral subtraction is used in which an average noise spectrum is estimated and subtracted from the signal (amplifier sensor data) spectrum. This improves the average signal-to-noise ratio (SNR) in the vibration signal. Assuming that the signal is distorted by a wide-band stationary additive noise, the following steps are used. A spectrogram is calculated over noise data, a mean and variance are calculated over spectrogram of the noise (in frequency), a threshold is calculated based upon the statistics of the noise, a spectrogram is calculated over the signal and a mask is computed by comparing the signal spectrogram to the noise threshold. Ultimately, the de-noised output signal is a derived using a mask.

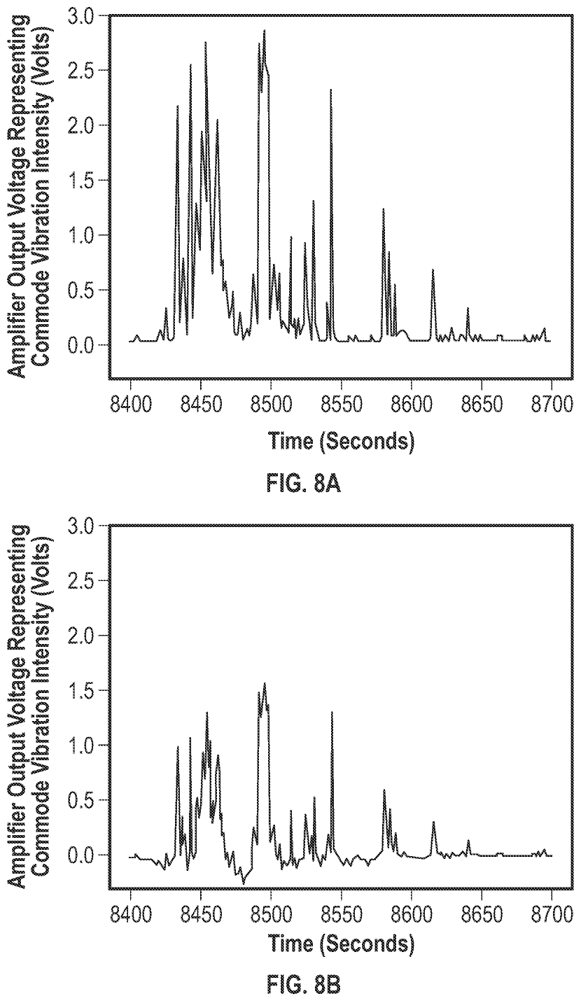

Referring now to FIGS. 8A and 8B, an example de-noising results from the above process shown in FIG. 7 is set forth. It can be observed that even though the amplitude of the signal reduces significantly due to spectral subtraction, the shape of the waveform is well-maintained. As discussed later, such shape maintenance is leveraged for identifying urination episodes and individuals involved with the episodes even in the presence of the noise. In FIG. 8A, a signal without spectral subtraction is illustrated. In FIG. 8B, an amplifier signal with spectral subtraction is illustrated.

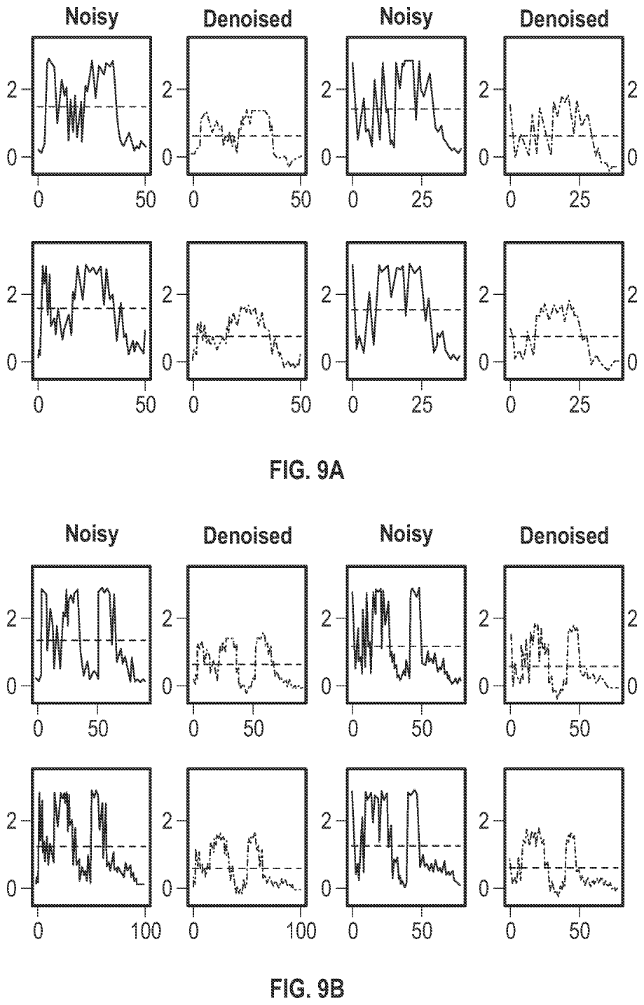

Referring now to FIGS. 9A and 9B, a plurality of urination signals, noised and de-noised, are illustrated without flush events. In FIG. 9B, urination signatures are provided with flush events, both noisy and de-noised.

Details of steps 714 through 718 are illustrated. In step 714, the urination episodes are identified and extracted. In step 716, the signals are demodulated to form a denoised and demodulated signal. In step 718, specific urinating individuals are identified based on the denoised and demodulated signal.

After de-noising the sensor data, the next step is to determine the urination episodes. From the data shown for different urination episodes in FIGS. 9A and 9B, it can be seen that a standard urination event consists of intermittent aperiodic peaks followed by a distinct signature of flushing and tank refilling. It can be seen that the events for flushing have very similar contour across all urination episodes. This commode flushing signature is used for extracting and labelling the urination events.

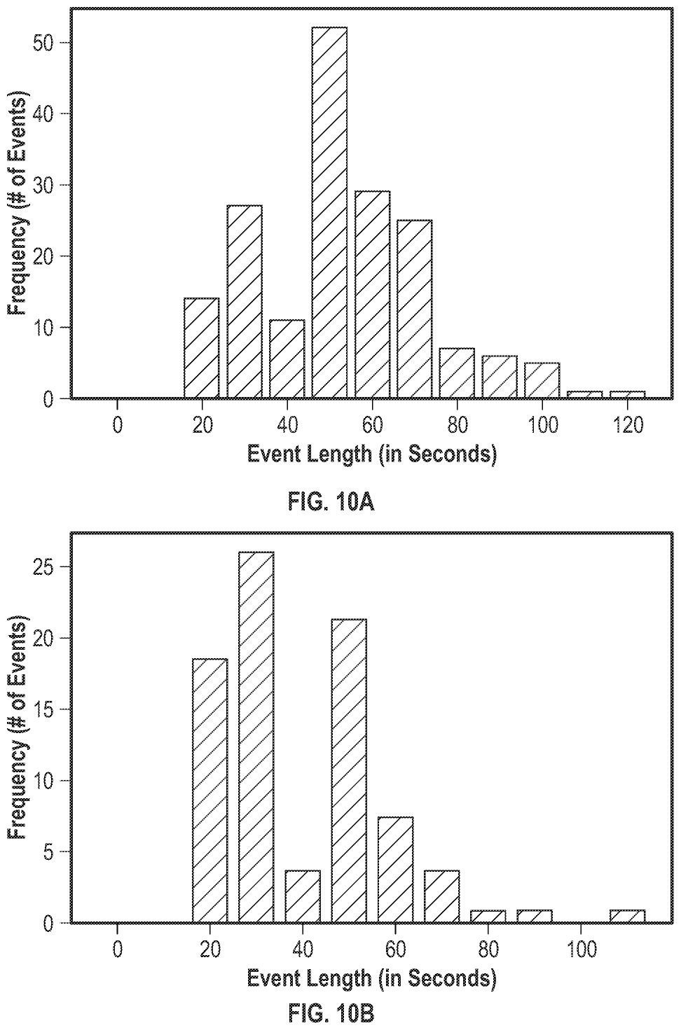

Referring now to FIGS. 10A and 10B, a sensor system from FIG. 1 was installed for a house-hold commode that was used by two subjects, one male and one female, for the urination dataset presented in the following sections. Sensor signal was continuously collected over a period of few days. The commode-flush signature, as described in the previous section, was then used for manually locating, extracting, and labelling about 180 urination episodes for subject-1, and about 90 episodes for subject-2. Both subjects also kept written records so that the extracted time-stamped episodes could be verified to be true urinary episodes and could be labelled for the specific subject. Such labels are used as ground-truths for the classification details presented in the next section.

The duration distribution of urination episodes from subject 1 is set forth in FIG. 10A. In FIG. 10B, the duration distribution of all the labelled urination episodes for subject 2 is set forth.

Machine learning may be used for determining the urination episodes and the person detected.

The ability of machine learning (ML) based algorithms for identifying unlabeled urination episodes and detecting the subjects related to the episode was performed. Using the extraction and labelling method described in the previous section, a dataset containing 278 urination and 5000 ambient events. The latter represents vibration signals caused by non-urination related events and are present throughput the sensor output signal. The ML mechanisms are deployed for distinguishing the urination episodes from the ambient vibration signal. Signal segments from this dataset were used for both training and testing purposes.

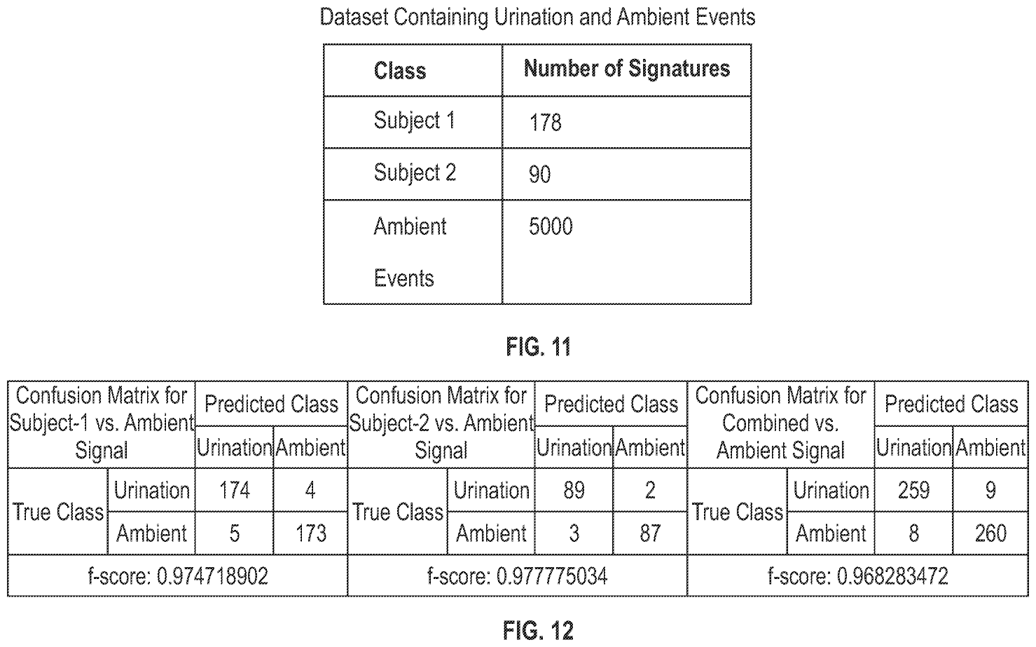

Referring now to FIG. 11, a neural network was designed for urination episode identification. Raw sensor data for 120 s (i.e., 120 samples collected at 1 Hz sampling rate) is fed to the network with 120 neurons at the input layer. Two hidden layers (i.e., with rectified linear unit (relu) activation layers), each with 200 neurons, feed to the output layer with two neurons, each signifying a class viz. urination episode and ambient event episode. Note that the 120 s duration is chosen based on the maximum length of the valid urination episodes. Zero padding is added to the end of any data item in FIG. 11 that is less than 120 s long. The classification results are presented below.

Urination episodes and ambient events are distinguished. Classification is done in both subject-specific and combined manner. To perform subject 1's urination episode classification, a data subset containing 178 urination and 178 ambient events is taken from the whole dataset specified in FIG. 11. Similarly, to perform subject-2's urination episode classification, a subset containing 90 urinations for subject-2 and 90 ambient events were taken. Finally, for the combined classification, 178 urinations for subject-1, 90 urinations for subject-2, and 268 ambient events were taken. Keeping the number of ambient events to be the same as the urination events keeps the dataset unbiased and makes classification results more reliable.

Referring now to FIG. 12, a classification performance across different subjects and on combined data. FIG. 12 shows classification performance for the urination episodes from individual subjects as well as on combined data. In addition to the low classification confusion, the results demonstrate that the system is able to classify urination with above 97% of f-score accuracy for individuals as well as for the combined data.

Once a urination episode is identified, the next processing step is to identify its subject as described in step 718. This is particularly useful when a single commode is used by my multiple individuals and the overall management needs to be targeted for one, many, or all of the individuals. For this, a neural network the input of which is the 120 samples of an already identified urination episode is used, and the two outputs correspond to the two subjects in this dataset.

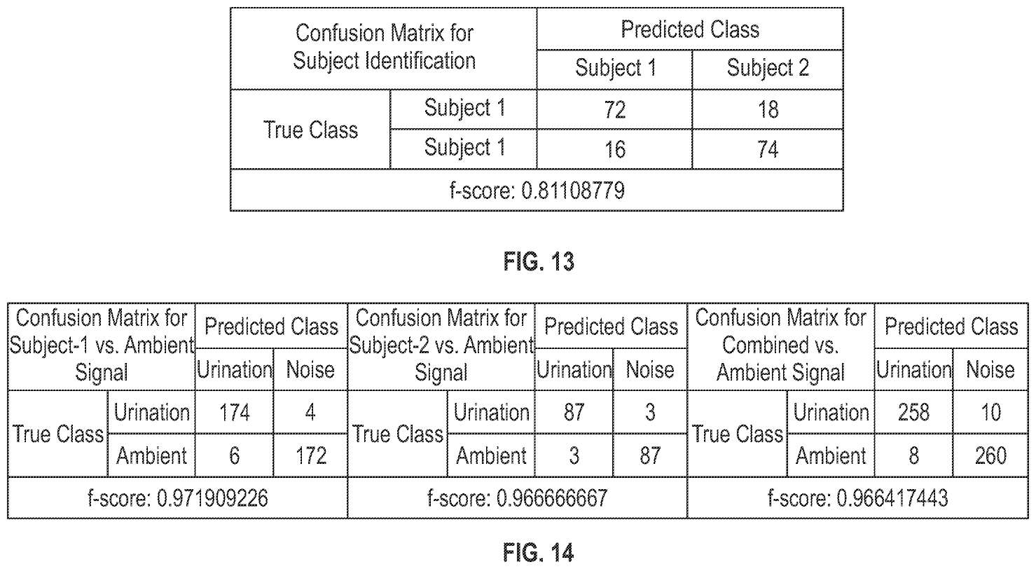

Referring now to FIG. 13, such subject classification performance, which provides an 81% f-score accuracy. Subjects 1 and 2 are wrongly attributed to 18 and 16 urination episodes respectively. This indicates that the deployed neural network configuration was not able to sufficiently distinguish between all the urination signatures across the two subjects.

The classification results in the previous section use all 120 samples (i.e., 120 s signal) as the input to the neural network. To handle such large input space, a very large neural network of size 120×200×200×2 is used. To make the model more amenable to a low computation microprocessor that can be incorporated within the sensor housing 40, the dimensions of the neural network architecture must be reduced. To reduce the input dimension from 120, various time domain features are engineered from those raw 120 samples. The features are selected such that characteristics of the urination episodes are preserved. Various time-domain features may be determined such as but not limited to, such as:

$$Mean Absolute Value ; {μ}_{av} = \frac{1}{N} {\sum }_{k = 1}^{N} \vert x ( k ) \vert \\ Mean ; μ = \frac{1}{N} {\sum }_{k = 1}^{N} x ( k ) \\ Variance ; var = \frac{1}{N} {\sum }_{k = 1}^{N} {( x ( k ) - μ )}^{2} \\ Energy ; E = \frac{1}{N} {\sum }_{k = 1}^{N} {( x ( k ) )}^{2} \\ Average Absolute Difference ; {μ}_{ad} = \frac{1}{N - 1} {\sum }_{k = 2}^{N} \vert x ( k ) - x ( k - 1 ) \vert \\ Mean Crossing \\ Skewness ; Skew = \frac{\frac{1}{N} {\sum }_{k = 1}^{N} {( x ( k ) - μ )}^{3}}{{[ \frac{1}{N} {\sum }_{k = 1}^{N} {( x ( k ) - μ )}^{2} ]}^{\frac{3}{2}}} \\ Kurtosis ; Kurt = \frac{\frac{1}{N} {\sum }_{k = 1}^{N} {( x ( k ) - μ )}^{4}}{{[ \frac{1}{N} {\sum }_{k = 1}^{N} {( x ( k ) - μ )}^{2} ]}^{2}} \\ Root Mean Square ; RMS = \sqrt{\frac{1}{N} {\sum }_{k = 1}^{N} {( x ( k ) )}^{2}} \\ Slope Sign Change \\ log - Detector ; log Detect = {e}^{\frac{1}{N} {\sum }_{k = 1}^{N} log ( \vert x ( k ) \vert )}$$

If the signal is product of actual signal and noise where;

x=Event/Noise (Sensor data)

N=Event length

The above feature set is first decomposed to calculate the principal components. Top ‘r’ subset of principal components is then chosen as features to train the urination identification model. The feature dimension the reduces from 120 to ‘r’. For the following experiment r=7, which makes the number of neurons in the input layer to be 7. Two hidden layers of 10 neurons each are used which are followed by rectified linear unit (relu) activation layers. The output layer has 2 neurons as before. This reduces the model size from 120×200×200×2 to 7×10×10×2, which is significantly simpler and more suitable for implementing within a microcontroller.

Referring now to FIG. 14, urination episodes vs. ambient events are distinguished by the classifier. Classification results from this new reduced architecture with specific feature engineering are presented. Classification performance across different subjects and on combined data using the reduced neural network was performed.

One observation from FIG. 14 is that the new reduced architecture approach reduces the overall f-score accuracies very slightly compared to the full architecture case in FIG. 12. However, the accuracy numbers are still above 96% across all three evaluated scenarios.

Referring now to FIG. 14 the subject may be Identified based on urination signatures. A set of results for subject distinction with the neural network architecture in the classifier is set for the in FIG. 14. FIG. 14 shows some improvement of the overall f-score accuracy, which is roughly 84.4% as opposed to the 81.1% achieved via the large network.

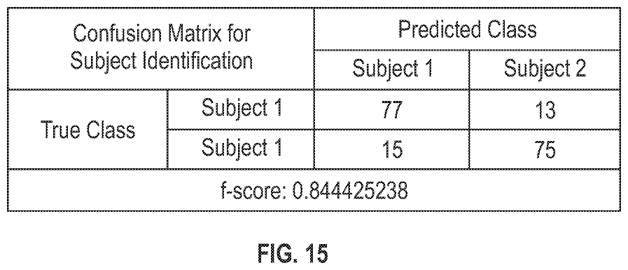

Referring now to FIG. 15, classification performance with the reduced network for subject identification is set forth. In summary, the results in FIGS. 14 and 15 demonstrate urination and subject classification using the reduced neural network can deliver a higher subject identification accuracy at the cost of very slightly lower urination episode detection accuracy, when compared to the larger network without feature engineering. These observations are promising in terms of being able to implement the classification software within the embedded micro-processor within the sensor box as shown above.

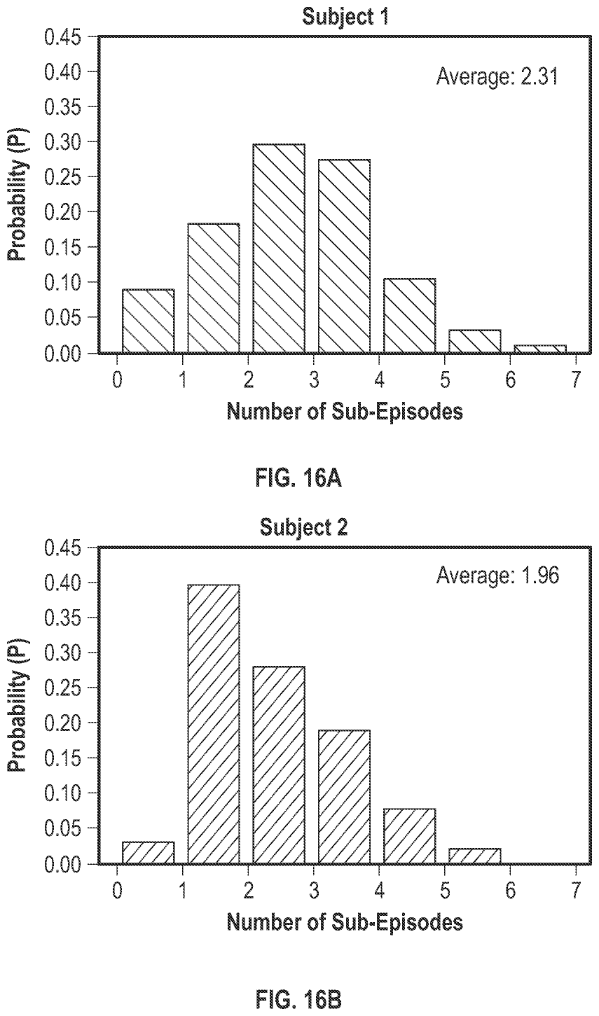

Referring now to FIGS. 16A and 16B, a urination sub-episode analysis for understanding bladder emptying patterns is set forth for each subject respectively. After the urination episodes and their subjects are identified, the next step is to analyze the episodes and their sub-episodes in order to get better insight into the urination process of a subject. Lowpass filtering followed by peak detection methods is used for identifying the sub-episodes. Example sub-episodes can be observed as described above. The urination episode contains three district sub-episodes, each representing a bladder emptying session within a urination session.

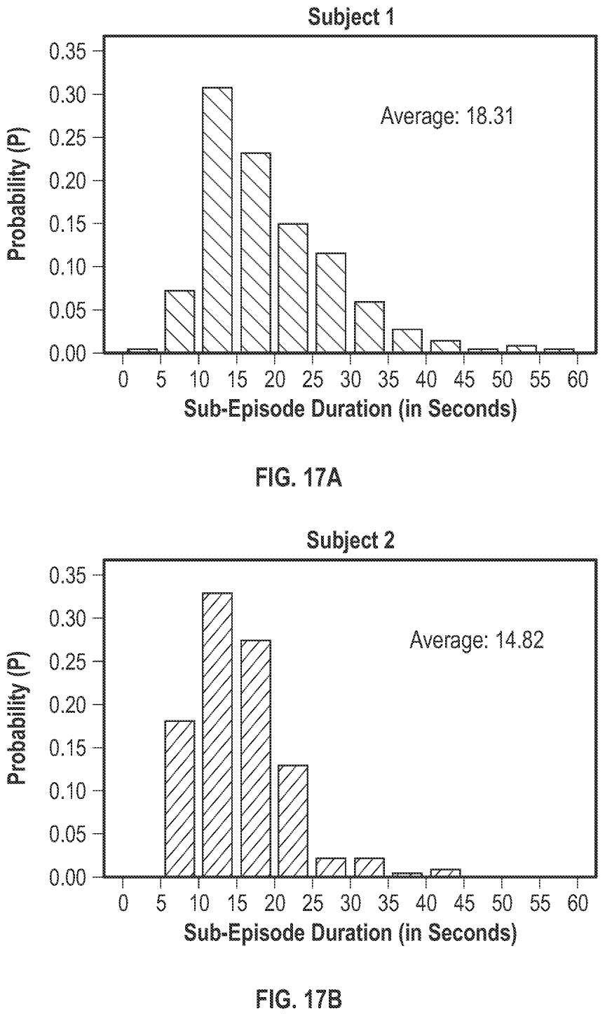

Referring now to FIGS. 17A and 17B, statistics of the number of sub-episodes in each urination episode are determined to depict the sub-event count statistics for the two subjects, respectively. The results correspond to 178 urination episodes for subject-1 and 90 urination episodes for subject-2. It can be observed that in general, subject-1 used a greater number of bladder emptying sub-episodes compared to subject-2. The average number for sub-episodes for the subjects are 2.31 and 1.96 respectively.

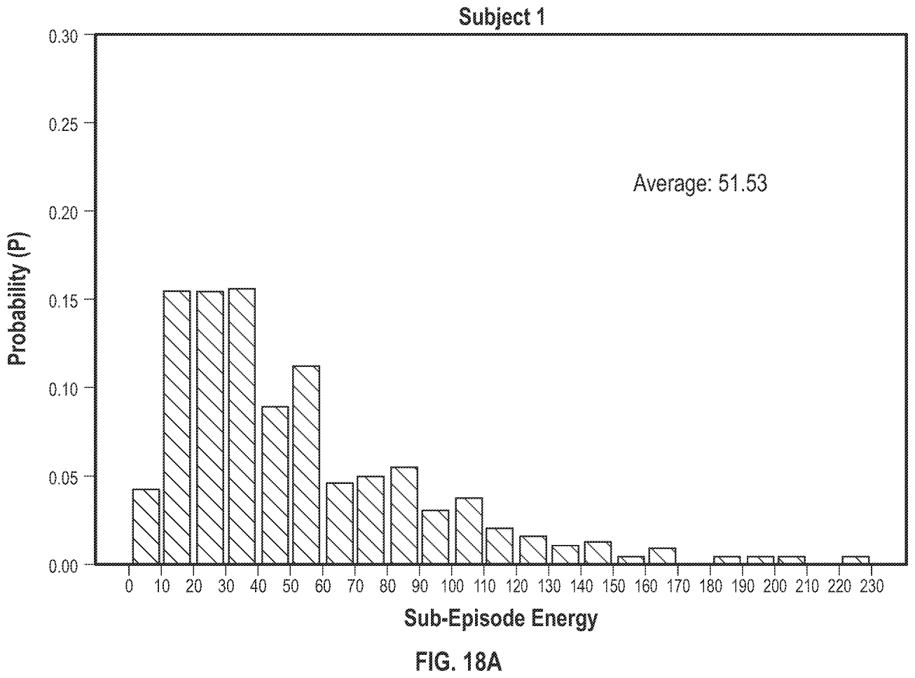

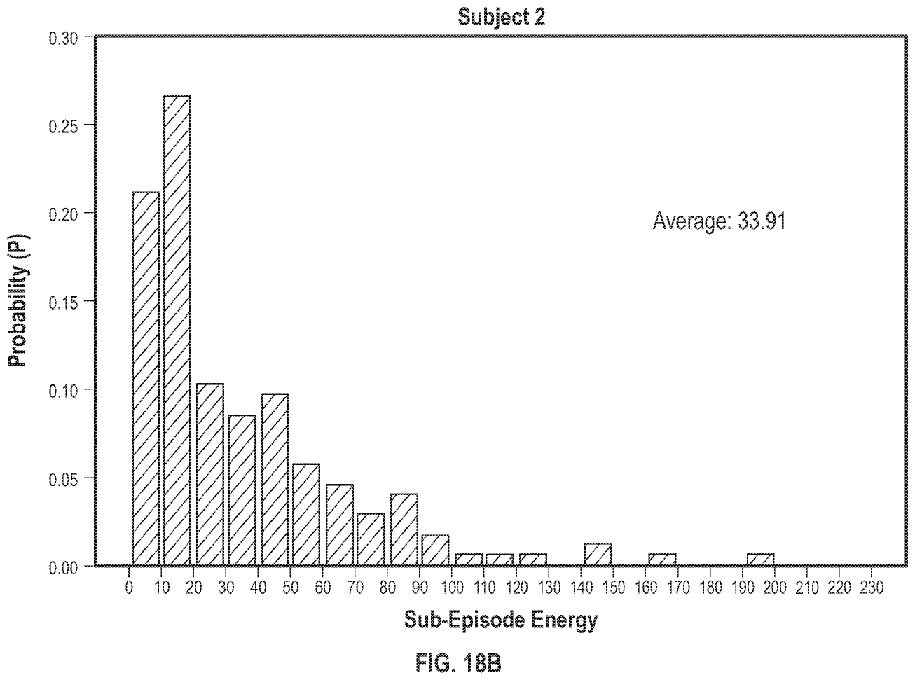

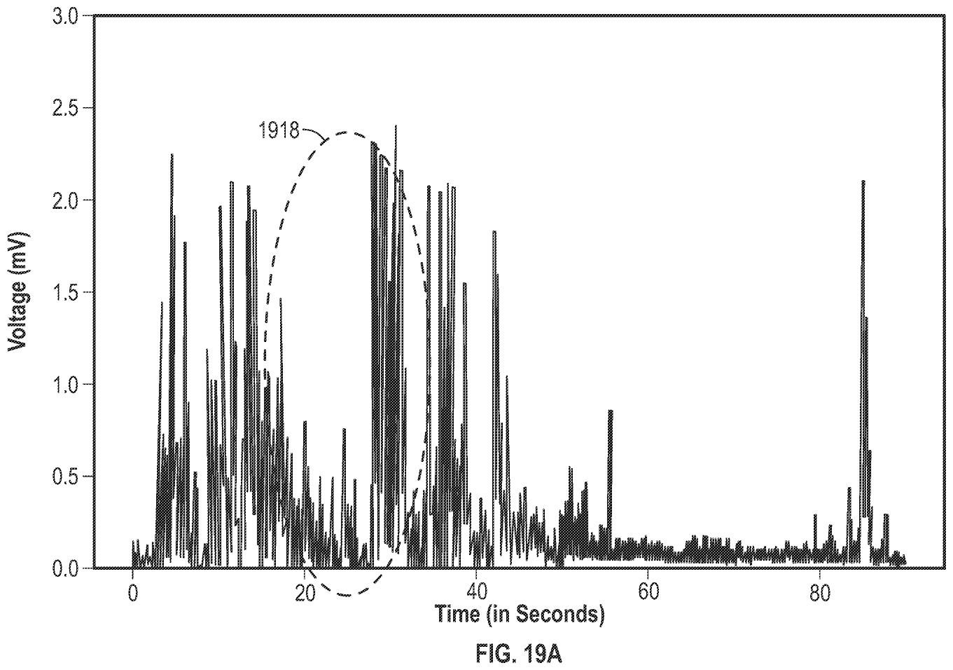

In FIGS. 18A and 18B sub-episode duration distribution statistics for each subject are determined. Much like the sub-episode duration, generally, the duration of each sub-episode for subject-1 is longer compared to those of subject-2. The average sub-episode durations for the two subjects are 18.31 seconds and 14.82 seconds respectively. As shown in FIGS. 10A and 10B, the episode durations for subject-1 were also longer than those for subject-2. Note that the distributions in FIG. 18 are created from a sample size total of 404 sub-episodes for subject-1 and 175 sub-episodes for subject-2.

FIGS. 18A and 18B ultimately shows the distribution of flow energy observed for the sub-episodes for both the subjects, respectively. For each sub-episode, energy is computed as:

$$E = \frac{1}{N} {\sum }_{k = 1}^{N} {( \vert x ( k ) \vert )}^{2} ,$$

where x(k) represents the k^{th} sample value, and N is the total number of samples within a sub-episode. The distribution and the average were computed over 404 sub-episodes for subject-1 and 175 sub-episodes for subject-2. The figure shows that the energy for subject-1's urination process is generally higher than those for subject-2.

Frequency domain signal analysis may also be used to analyze the output of the sensor. Frequency domain analysis is a mathematical technique used to analyze signals or data in terms of their frequency components. It involves transforming time-domain data into the frequency domain to reveal the underlying spectral characteristics of a signal. Instead of examining how a signal changes over time, it focuses on the different frequencies that make up the signal and their respective amplitudes. It transforms a signal from the time domain, where data is represented as amplitude values over time, into the frequency domain, where data is represented as amplitude values across different frequencies. In the context of a vibration cantilever-based sensor generating time series voltage data, it helps pinpoint the frequencies associated with vibrations or mechanical oscillations.

Performing frequency domain analysis for urinary signature analysis provides a number of benefits. Frequency domain analysis may be used to capture complex vibrations. When the sensor generates complex vibrations with multiple frequency components, time-domain analysis may not adequately capture the underlying patterns. Frequency analysis excels at decomposing such signals into their constituent frequencies, providing a clearer understanding of the sensor's behavior.

Frequency domain analysis may also be used for resonance identification: Frequency analysis is excellent for identifying resonance frequencies, where the sensor's response is most pronounced. This property is crucial for specific applications like urination detection. Resonance frequencies can be masked or challenging to discern in the time domain.

Further frequency domain analysis may be used for weak signal analysis. Frequency domain analysis excels when dealing with weak signals, such as urination on the solid part of the commode as opposed to in the water in it. In this scenario, time-domain analysis struggles to distinguish the urination event due to the low signal amplitude. However, frequency domain analysis is found to be able to isolate the characteristic frequencies associated with urination, allowing for more robust detection and analysis.

In scenarios where sensor data is contaminated with noise or interference (especially multiple frequency noise), frequency analysis can effectively filter out unwanted frequencies, enhancing the precision of information extraction.

Frequency domain analysis pipeline for the urination sensor signal may be performed in many ways individually or in combination. Bandpass filtering is one technique that may be used. Bandpass filtering is a signal processing technique that selectively passes frequencies within a specified range while attenuating those outside the range. It is often used to isolate specific frequency components of interest from a broader spectrum of frequencies. By using bandpass filtering, the time-domain signal may be retained with the urination event isolated.

Spectrogram extraction is another technique. For analysis of the sensor signal. A spectrogram is a visual representation of the time-varying frequency content of a signal. It displays how the amplitudes of different frequency components change over time. It provides a two-dimensional plot with time on the x-axis, frequency on the y-axis, and color indicating the magnitude of each frequency component at a given time. In the context of urination event detection, spectrograms can provide insights into how the identified frequency ranges evolve during urination.

Wavelet scalogram is another technique for analyzing the urination signal. A wavelet scalogram is a time-frequency representation of a signal created using wavelet transforms. Wavelet transforms are versatile tools for analyzing signals with varying frequency content at different scales. Wavelet scalograms provide insights into how frequency components change over time. It allows for the analysis of signal components at different scales and time instances. In the context of the sensor monitoring urination events, the wavelet scalogram can offer a detailed view of how the identified frequencies vary over time.

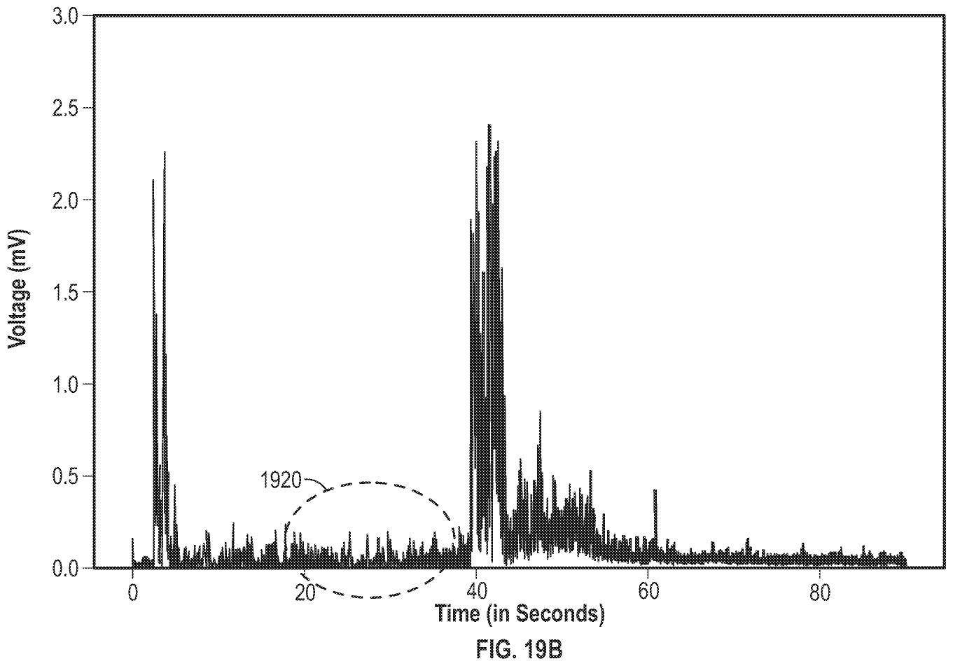

Referring now to FIG. 19A, experimentation with frequency domain analysis of urination sensor signal has been performed for two types of urination signals recorded from the sensor and analyzed. On-Water Urination is the first type. Urine flow is directed on the in-commode water. Due to the forced direct contact of urine, the sensor records high amplitude vibrations. This is due to the liquid's ability to transmit vibrations efficiently which leads to high output voltages being recorded at the sensor. One example recorded signal is shown below. The circled region 1910 is the on-water urination event signal. To be noted that the relatively flat portion following the urination event is the flush followed by the attenuating portion of the signal which is the tank refilling.

The second type of urination signal is on-solid Urination. Urine flow is directed on the inner solid of the commode, the sensor detects low amplitude vibrations, as the solid attenuates the vibrations significantly. This leads to low output voltages being recorded at the sensor. An instance of the recorder on-solid signal is shown below.

The circled area 1920 is the on-solid urination event signal. The location of flush and tank refilling portion on the time-axis is very similar to the on-water urination signal. To be noted that the amplitude of urination event is comparatively very low as compared to the on-water urination event.

Processing in frequency domain may be performed. To locate the frequency components that carry most of the information related to urination, spectrogram and wavelet scalogram are used. After determining the frequency ranges, bandpass filtering is applied, and then corresponding time-domain signal is reconstructed to verify the existence of urination events.

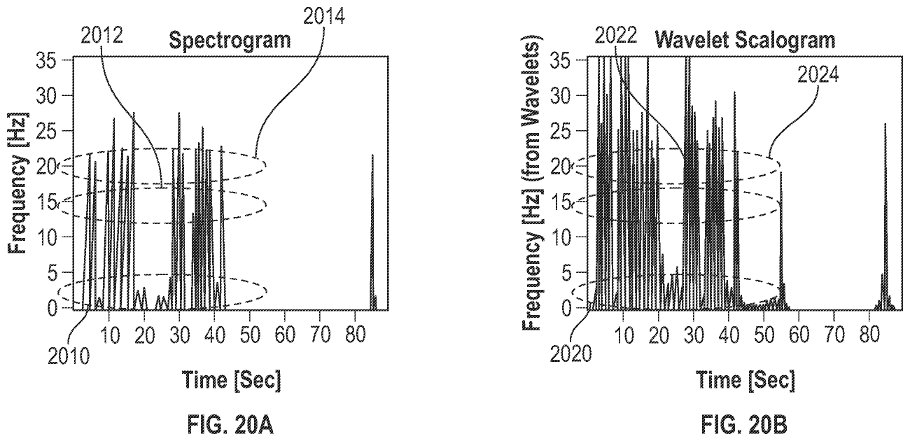

Time-Frequency Representation Spectrogram and wavelet scalogram of both types of signals are extracted to locate the dominant frequency constituents of the signals. The frequency ranges are viewed in the time-varying frequency representation in FIG. 20A as a spectrogram and in FIG. 20B as a scalogram for the on-water example shown in FIGS. 20A and 20B. Both, spectrogram and scalogram plots, unveil that the most significant magnitude in the recorded data is concentrated in frequency bands of 0.5-3 Hz, 14-15 Hz, and 19-21 Hz as illustrated respectively by the circled areas 2010, 2012, 2014 in the spectrogram of FIG. 20A and as illustrated respectively by the circled areas 2020, 2022, 2024 in the scalogram of FIG. 20B.

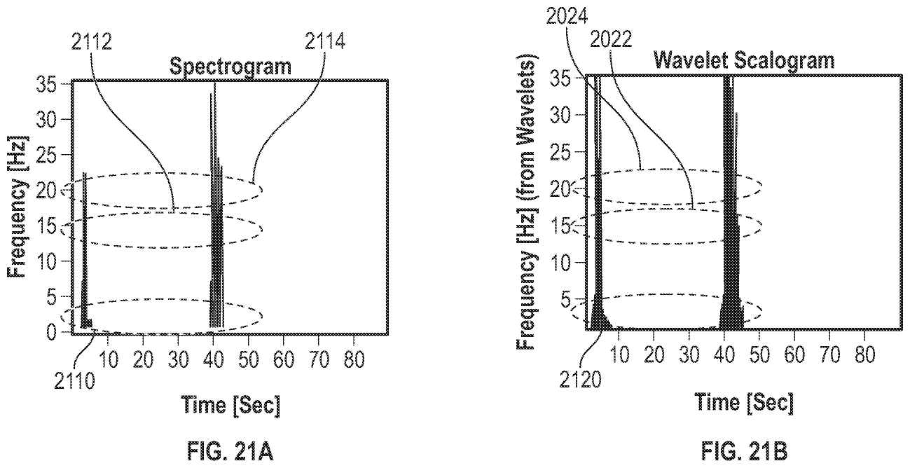

Referring now to FIGS. 21A and 21B a spectrogram and a scalogram of on-solid urination are set forth. The plots are for the representative example of on-solid urination. Like the previous scenario, in this example scenario, the spectrogram showed that the same frequency ranges (0.5-3 Hz, 14-15 Hz, and 19-21 Hz) had high magnitude during urination events as illustrated respectively by the circled areas 2110, 2112, 2114 in the spectrogram of FIG. 21A and as illustrated respectively by the circled areas 2120, 2122, 2124 in the scalogram of FIG. 21B.

For this operation, the frequencies of interest were 0.5-3 Hz, 13-15 Hz, and 19-21 Hz, according to the outcomes of spectrogram and wavelet scalogram. It is observed that even after using bandpass filtering, the time-domain signal was retained with the urination event.

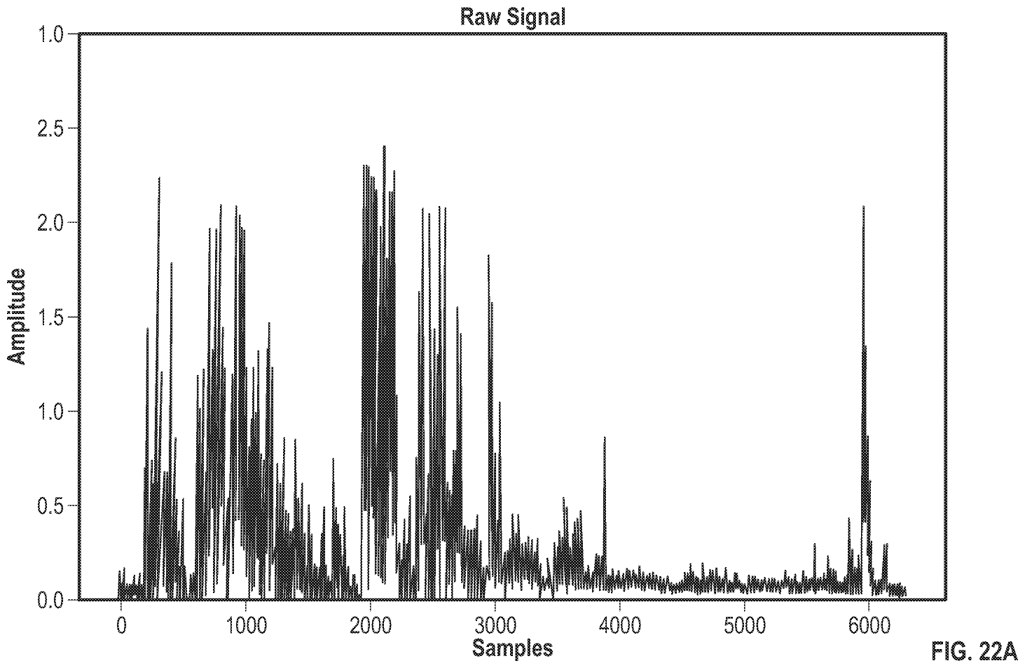

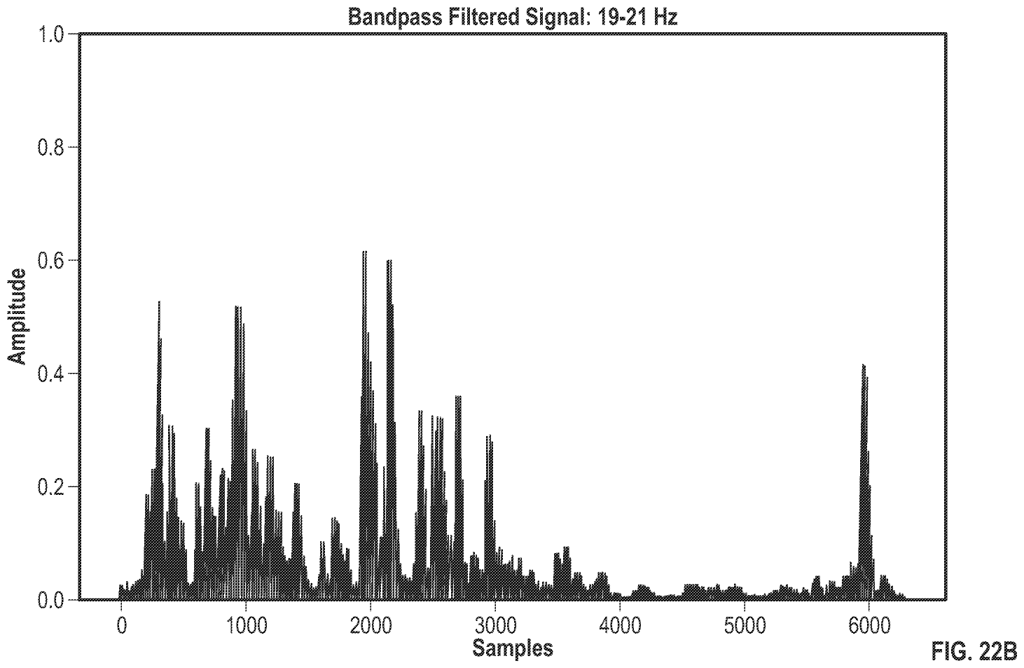

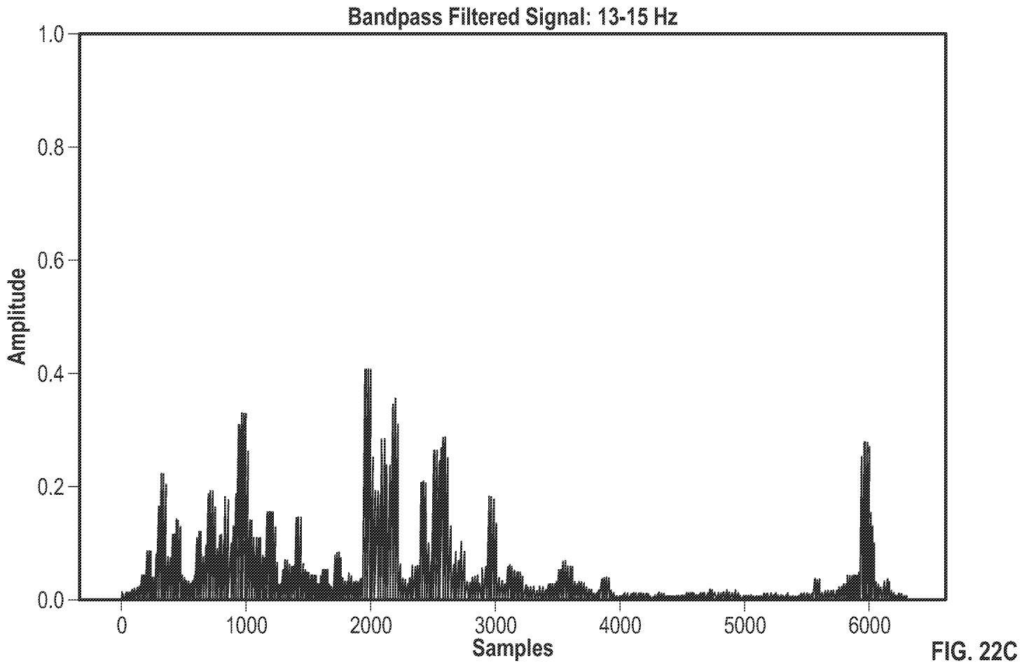

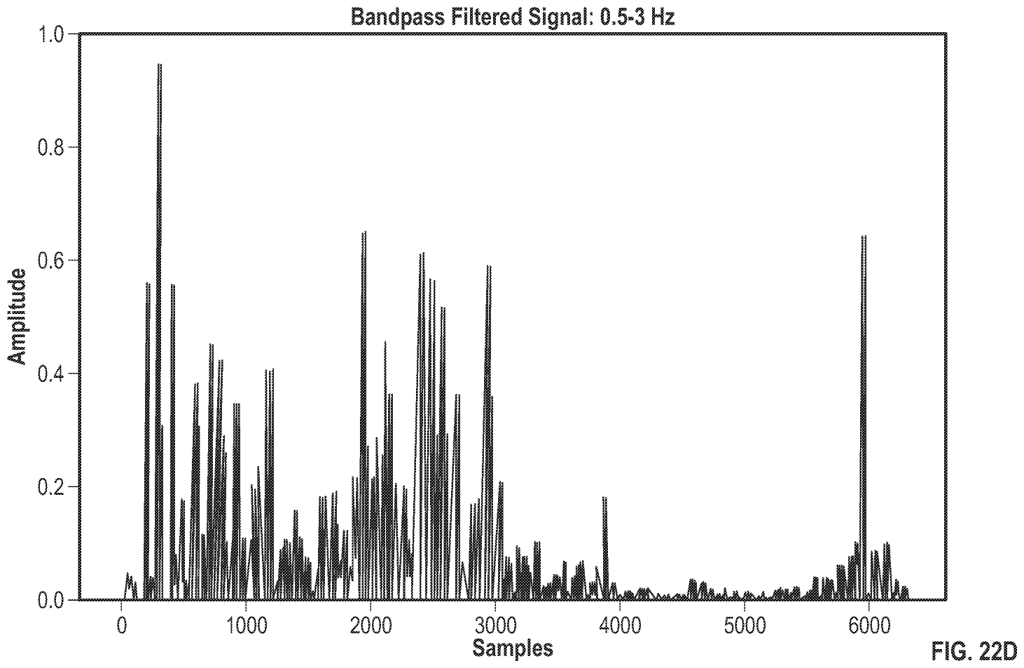

Referring now to FIGS. 22A-D and 23A-D, a raw signal and bandpass filtering of an on-water urination event is set forth with FIG. 22A showing an on-water time domain signal of a raw signal of an event, FIG. 22B showing the signal of FIG. 22A band pass filtered in the 19-21 Hz range, FIG. 22C showing the signal of FIG. 22A band pass filtered in the 12-15 Hz range and FIG. 22D showing the signal of FIG. 22A band pass filtered in the 0.5-3 Hz range.

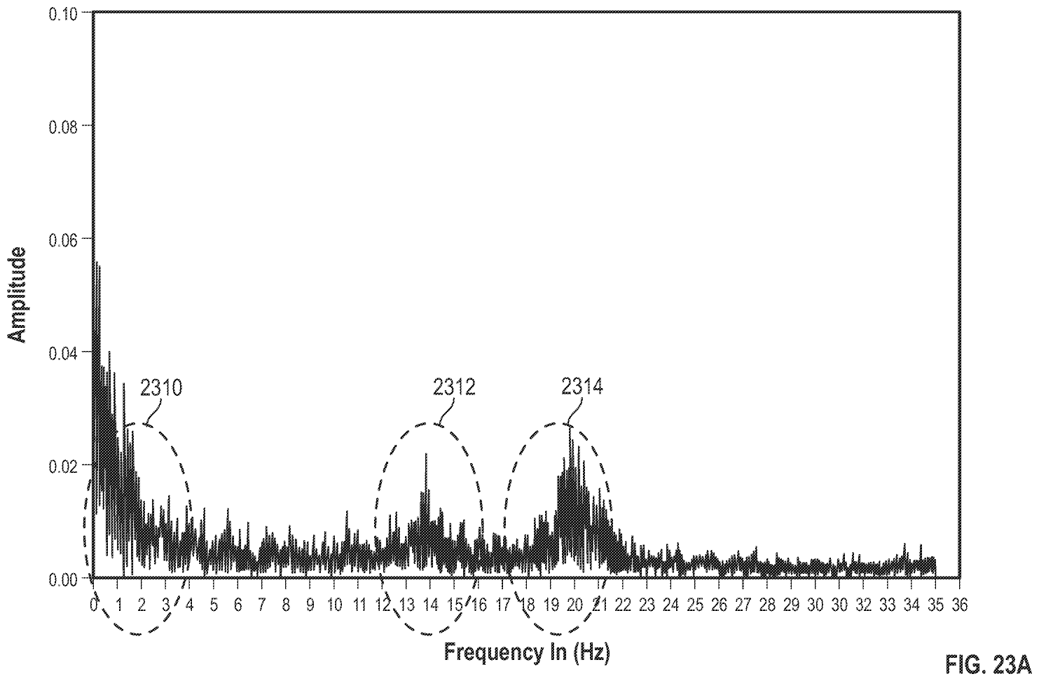

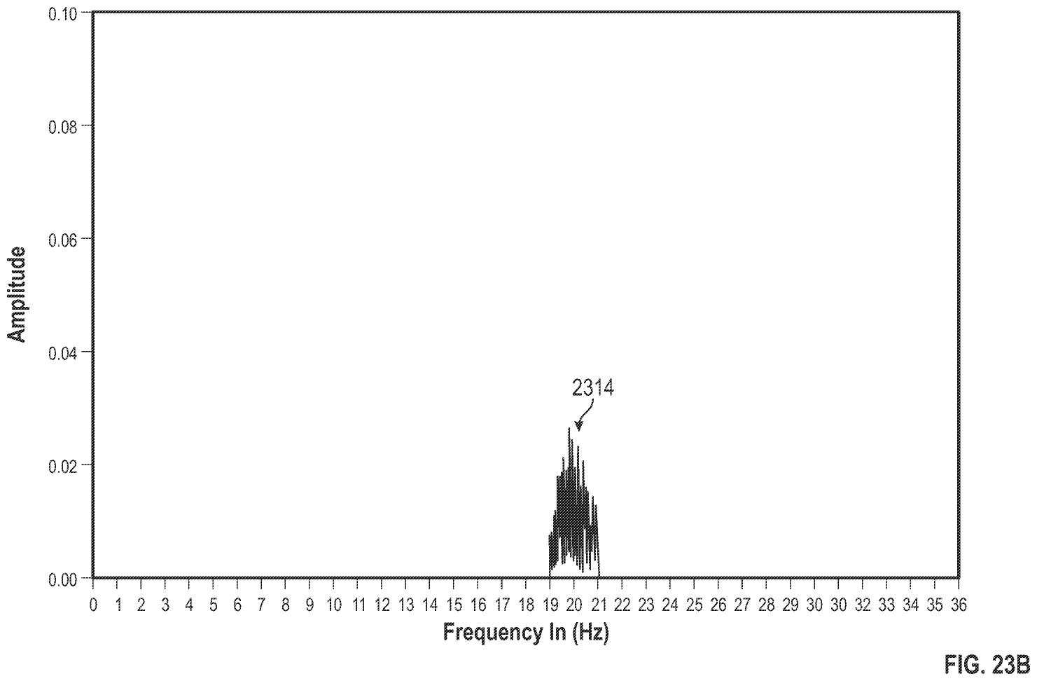

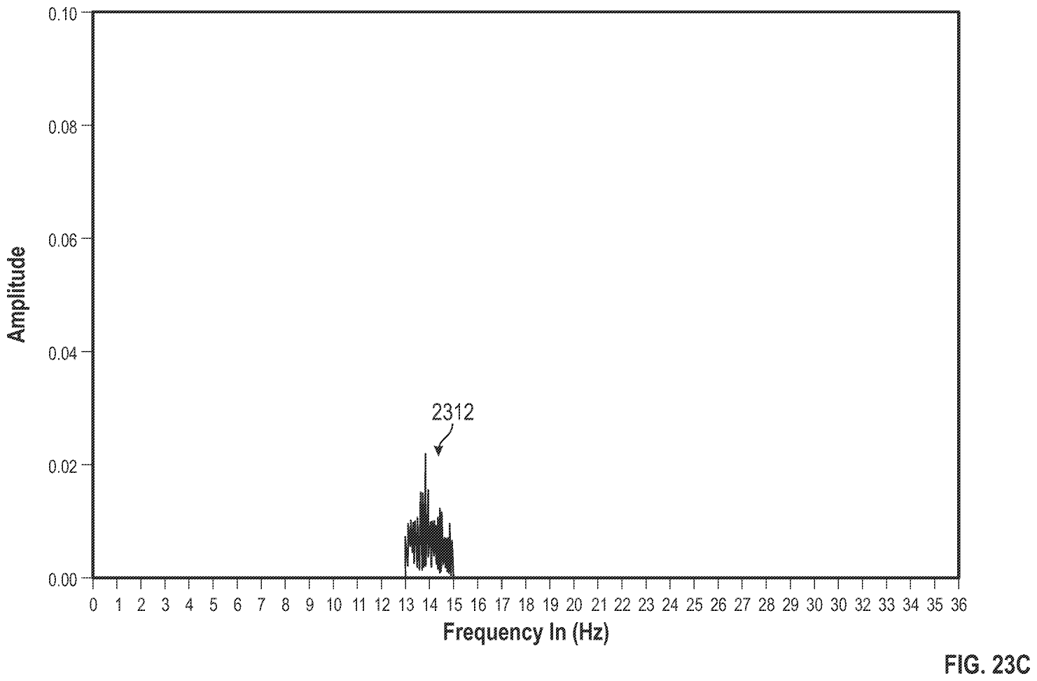

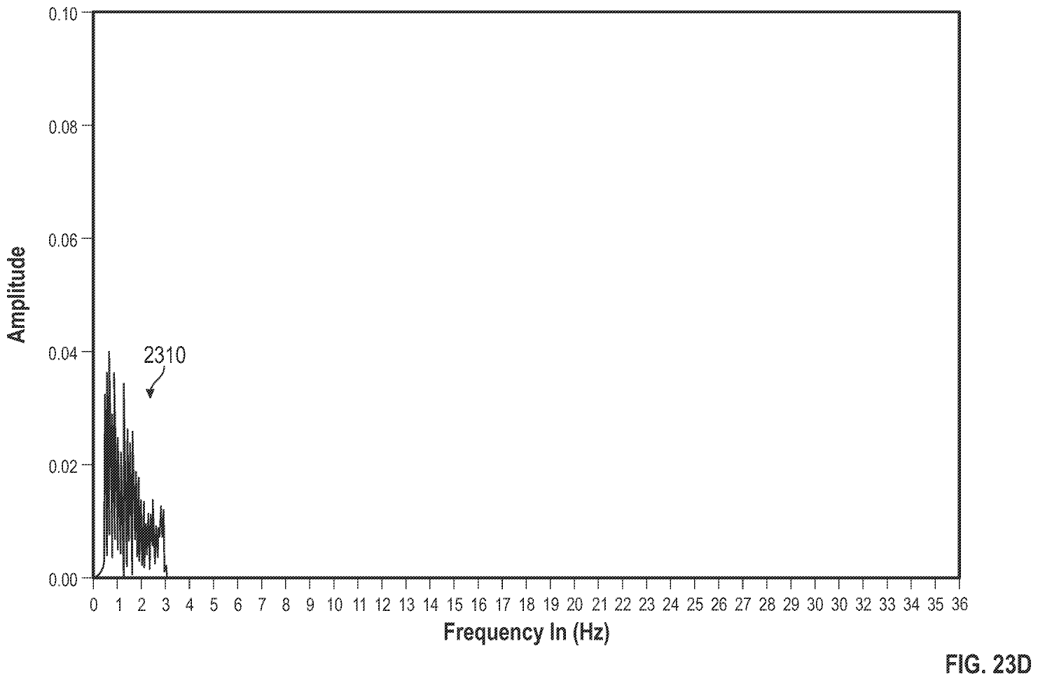

Referring now to FIGS. 23A-D, similar raw and filtered signals are illustrated in the frequency domain. Bandpass filtering is employed to extract the relevant signal components within the identified frequency bands (0.5-3 Hz (FIG. 23D at 2310), 13-15 Hz (FIG. 23C at 2312), and 19-21 Hz (FIG. 23B at 2314) while eliminating unwanted frequencies. This process retains the urination events in the reconstructed time domain signal.

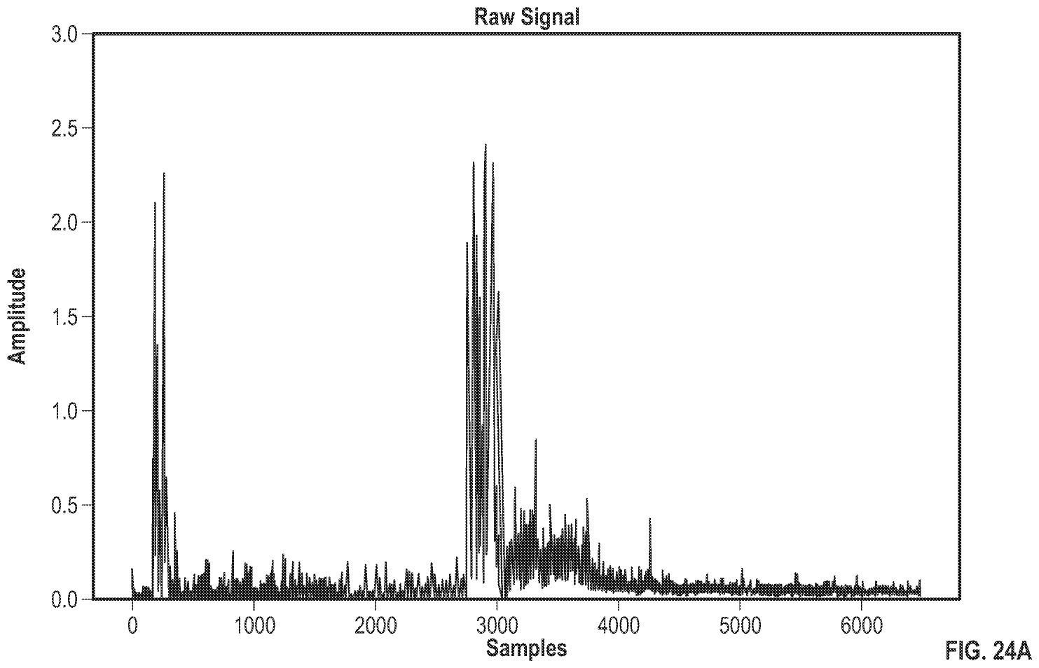

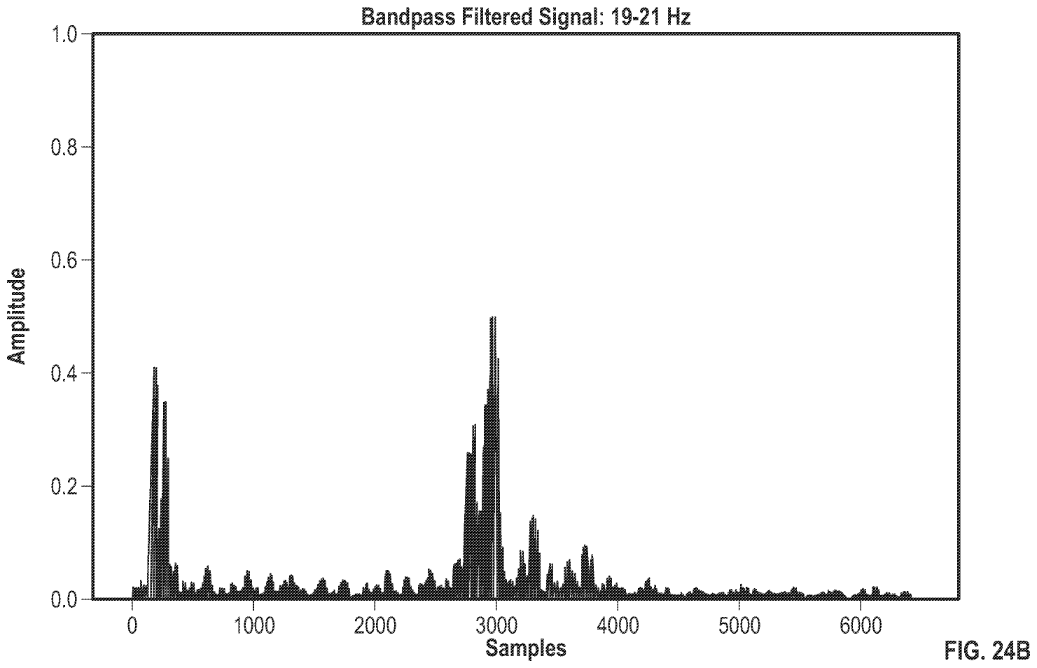

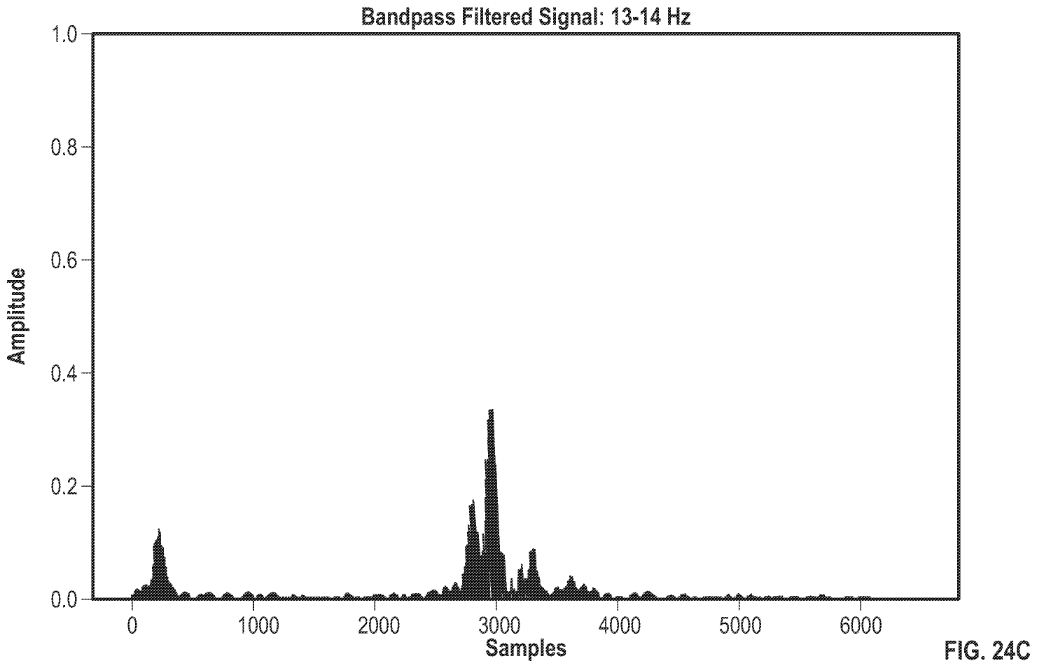

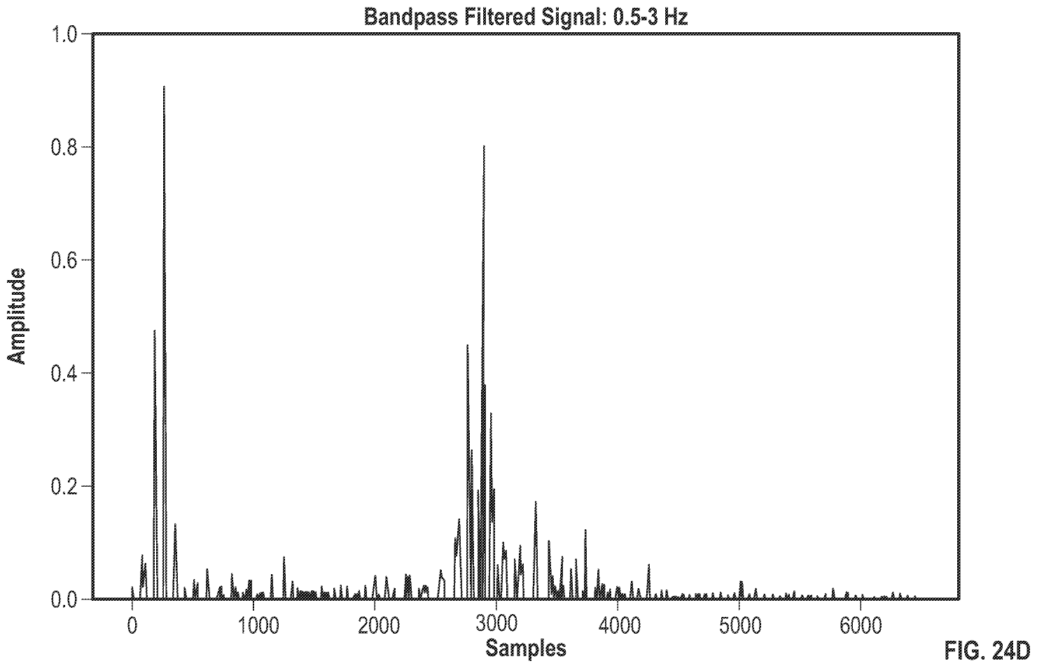

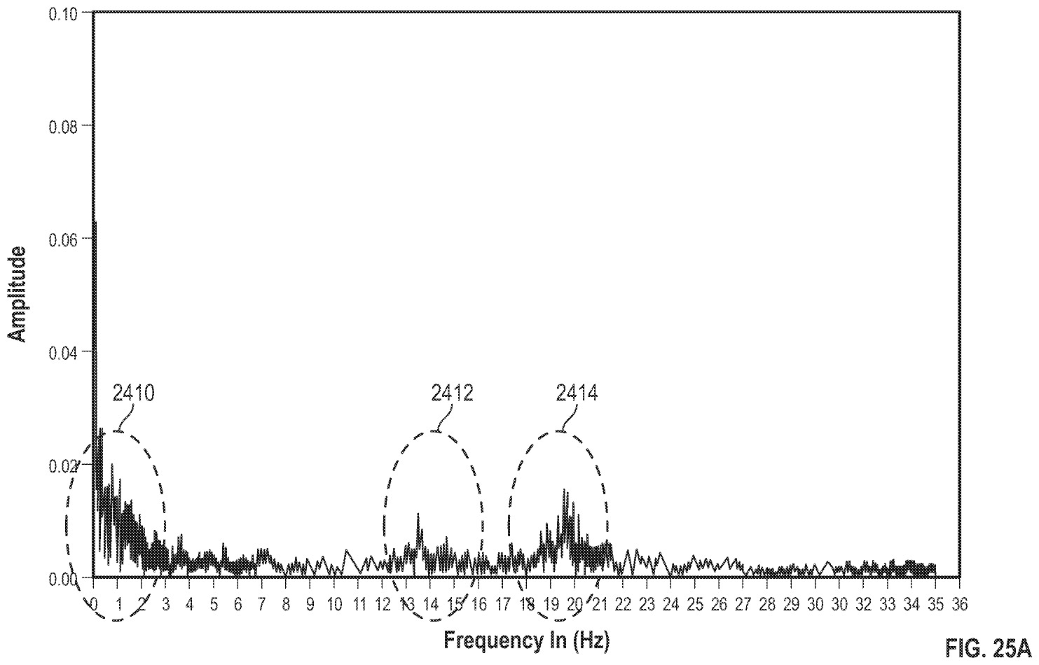

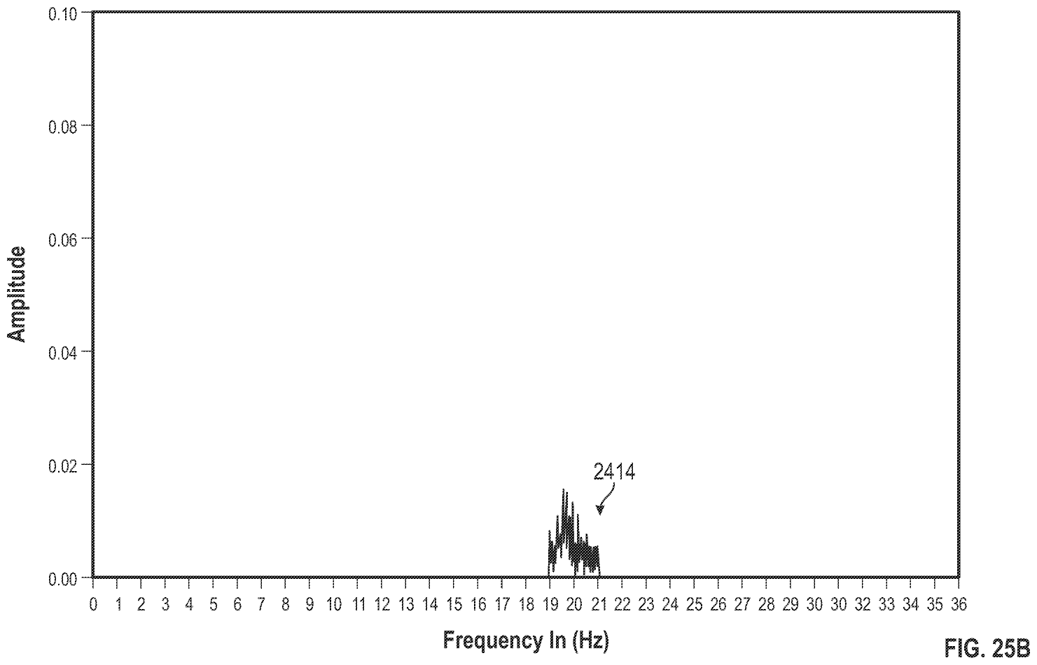

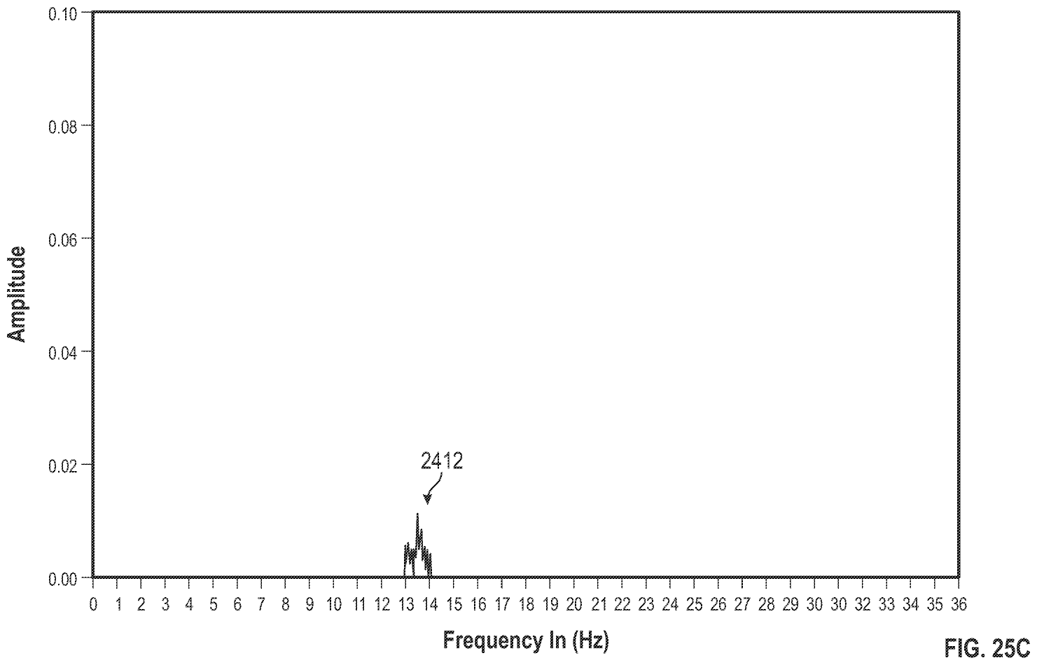

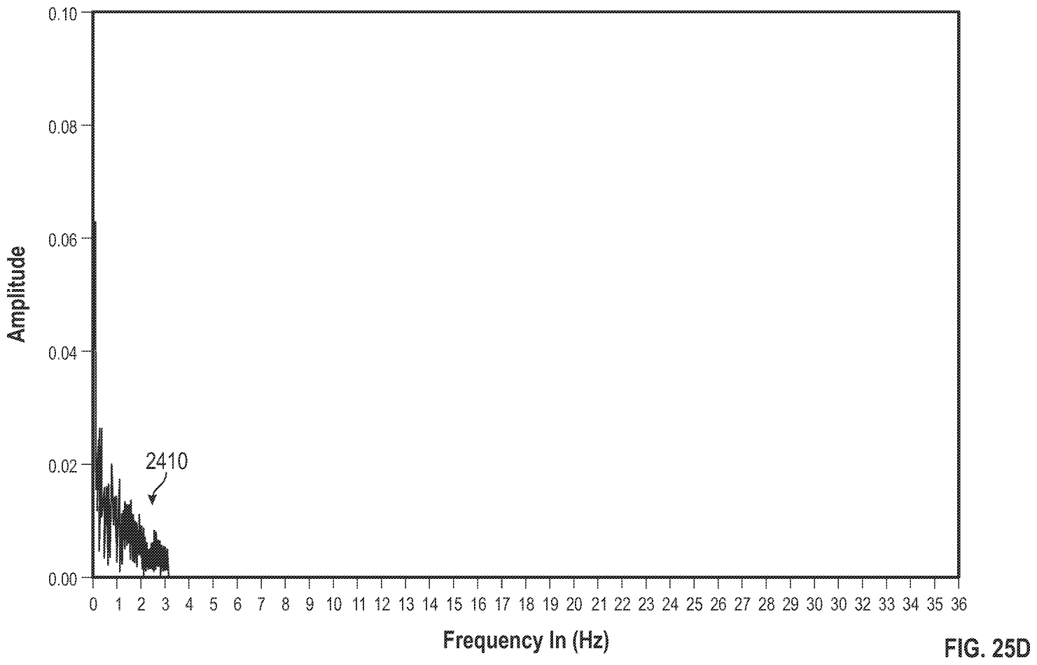

Referring now to FIGS. 24A-D, a similar manner to the above on-solid raw signals is obtained and filtered. In the on-solid scenario described, bandpass filtering is used to isolate the relevant frequency components (0.5-3 Hz, 13-14 Hz, and 19-21 Hz) from the raw signal (FIG. 24A) associated with urination events while eliminating unwanted noise. Unlike the bandpass filtering of on-water signal, the urination event is retained in the reconstructed time-domain version for frequency ranges 0.5-3 Hz at 2410, and 19-21 Hz at 2414. However, for the range 13-14 Hz at 2412, the reconstructed time-domain version doesn't have substantial amplitude.

The results from processing in frequency domain may be interpreted to obtain an indication or urinary issues. Frequency domain analysis excels when dealing especially with weak signals, such as urination on a solid. In this scenario, time-domain analysis might struggle to distinguish the urination event due to the low signal amplitude. However, frequency domain analysis can isolate the characteristic frequencies associated with urination, allowing for more robust detection. Such inference is more apparent after the urination signature retrieval post-bandpass filtering, for both on-water and on-solid urination signal.

In conclusion, frequency domain analysis is a powerful tool for analyzing the vibrations recorded by a cantilever piezoelectric sensor in the context of urination detection. It helps identify relevant frequency components, enables event detection, and proves especially useful when dealing with weak signals or scenarios where specific frequency ranges are indicative of events of interest, such as urination on different surfaces. The combination of representation and transformation methods like bandpass filtering, spectrogram, and wavelet scalogram enhances the ability to accurately detect and differentiate urination events based on their distinct frequency signatures.

Frequency domain features may be exploited for urination signatures detection in signal from the vibration sensor. The following frequency-based features are used for such detection.

A first feature is dominant frequency. The dominant frequency represents the frequency component with the highest magnitude in the frequency domain analysis. It indicates the most prominent vibration frequency during urination. A higher dominant frequency may suggest a faster or more forceful urination event.

Another feature is frequency band energy in which the frequency spectrum is divided into specific frequency bands (e.g., low, medium, and high frequencies) and calculate the energy within each band. This feature provides information about the distribution of energy across different frequency ranges during urination.

Another useful feature is spectral entropy. Spectral entropy measures the randomness or complexity of the frequency distribution within the urination event. High spectral entropy indicates a wide distribution of frequencies, suggesting a complex event, while low entropy suggests a more concentrated frequency distribution.

Spectral kurtosis is another useful frequency feature. Spectral kurtosis quantifies the “tailedness” of the frequency distribution. A higher kurtosis implies a concentration of energy around specific frequencies, indicating distinct characteristics in the urination signature.

Frequency variability may also be used to analyze how the dominant frequency changes over the course of the urination event. Variability in the dominant frequency can reveal fluctuations in urination force or flow rate.

Identifying the person or subject performing the urination may be performed. Frequency domain features are used for subject identification based on their unique urination patterns. This is particularly useful when a commode is used by multiple individuals. Frequency features may be applied in this context in various way including frequency pattern matching by compare the frequency spectra of urination events for different subjects and identifying characteristic frequency peaks or patterns that are unique to each individual.

Another subject identification technique using frequency analysis is individual dominant frequencies by determining the dominant frequency for each subject's urination events. These individual dominant frequencies can serve as identifiers, as they may vary slightly from one person to another.

Yet another subject identification technique using frequency analysis is frequency band ratios. By calculating the ratios of energy or magnitude in specific frequency bands (e.g., low-frequency to high-frequency ratio), each subject may be identified. These ratios can capture subject-specific frequency characteristics.

Further spectral clustering may be used for subject identification. Clustering techniques maybe used to group urination events based on their frequency spectra. Subjects with similar frequency patterns will belong to the same cluster, facilitating subject identification.

Frequency analysis may also be used for estimating urination force by examining how the force-related characteristics manifest in the frequency domain. Urinary frequency-force correlation may be used. The correlation between the dominant frequency and urination force may be determined. A strong positive correlation may indicate that higher force results in a shift towards higher frequencies.

Urination force may be determined by spectral energy distribution by analyzing how the spectral energy is distributed across different frequency ranges. A change in energy distribution, such as an increase in higher-frequency energy, may correspond to increased urination force.

Frequency-based regression models may be used to determine urinary force. Regression models that predict urination force based on features derived from the frequency domain may be used. Features can include dominant frequency, spectral entropy, or frequency band energy ratios.

Dynamic frequency analysis is another technique to determine urinary force by monitoring how the frequency content of the sensor's signal changes throughout the urination event by tracking variations in the dominant frequency and spectral characteristics as force varies.

Multi-sensor fusion may also be used for urinary force determination by combining frequency-based features from the cantilever piezoelectric sensor with data from other sensors, such as pressure sensors or flow meters. Integrating multiple sensor modalities can enhance the accuracy of urination force estimation.

In summary, a system design, and preliminary results from an unobtrusive urination sensing, processing, and management framework is provided. An innovative and ultra-sensitive piezoelectric vibration sensor and machine learning algorithms are successfully used for high-accuracy urination and subject detection. It was also shown that urination episodes may be successfully analyzed for identifying sub-episodes and their many properties which can be used for clinical purposes.

From a technology development standpoint, the system may be miniaturized in terms of electronics for sensor signal processing. Wi-Fi connectivity to backend cloud system may be provided. Depending on the use, on-the-cloud algorithms and software may be developed for transforming sensor data and machine-learning detected parameters into clinically actionable information. Likewise, a mobile phone application for the user device may be used for accessing data and processing results from the backend cloud. Collaborating with urologists for developing a large dataset and their clinical implications may also be developed.

There are a plurality of clinical applications leveraging the data obtained by the present system. The data may be developed for diagnostics, disease progress, monitoring effects of prescription, and overall personalized treatment plan by the Urological health providers. Optionally, intervention feedback can also be provided to the patients for modifying voiding behavior, when applicable. Although the technology is not restricted to any specific demography, the initial commercialization of the proposed device/system will be targeted mainly towards elderly people staying at home, nursing home, and other residential settings. Possible diseases and conditions that may be determined include but are not limited to enlarged prostate, overactive bladder, nocturia, polyuria, incontinence management via predictive alerts, overall hydration management, predicting and managing urinary tract infection, water-economic flush volume based on the urination amount, water leakage detection due to faulty flap.

Remote diagnostics and monitoring services can be packaged along with the monitoring device itself. Clinics, nursing homes, and hospitals will be the customer base.

Non-medical products may also be formed from the present teachings. An environmentally-friendly commode flushing system may be provided. An amount of water used for a post-urination flush is decided based on the amount of urine discharge. The product can be sold as a standalone device, either integrated or retrofitted with the commode. The commode-attached device estimates the amount of urine discharge, and it controls the flush duration for the minimum required water dispensing, thus saving a significant amount of water wastage. Market size for this would be 100s of millions, covering many homes, offices, and public places such as airports, train stations etc.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A urination monitoring system for a commode comprising:
a housing coupled to an exterior of the commode;
a vibration sensor disposed within the housing generating a vibration signal corresponding to vibration of the exterior of the commode;
a signal shaping circuit coupled to the vibration sensor generating a processed vibration signal;
a controller comprising a trained classifier coupled to the sensor receiving the processed vibration signal and determining a urinary episode characteristic therefrom; and
an analyzer comprising a display, said analyzer coupled to the controller and generating an output corresponding to the urinary episode characteristic.

2. The urination monitoring system of claim 1 wherein the urinary episode comprises at least one of a urinary frequency, urinary flow strength and duration, a number of episodes within a urination episode, a low strength and duration of sub-episodes within the episode.

3. The urination monitoring system of claim 1 further comprising a commode wall and wherein the vibration sensor is coupled within the commode wall.

4. The urination monitoring system of claim 1 wherein the vibration sensor comprises a piezoelectric sensor.

5. The urination monitoring system of claim 4 wherein the piezoelectric sensor comprises a cantilever comprising a first end fixed within the housing at a base, said cantilever comprising a second end comprising a ballast, said sensor comprising a piezoelectric layer disposed between the ballast and the base.

6. The urination monitoring system of claim 1 wherein the signal shaping circuit comprises an amplifier and a signal de-noising circuit.

7. The urination monitoring system of claim 6 wherein the de-noising circuit comprises a band pass filter.

8. The urination monitoring system of claim 6 further comprising a demodulation circuit in communication with the signal shaping circuit and forming a denoised and demodulated signal, wherein the analyzer identifies a urinating individual based on the denoised and demodulated signal.

9. The urination monitoring system of claim 8 wherein the analyzer generates a flap leak signal based on the denoised and demodulated signal.

10. The urination monitoring system of claim 6 wherein the analyzer analyzes urinary episodes and sub-episodes based on energy therein.

11. A method for monitoring urination at a commode comprising:
generating a vibration signal corresponding to vibration from a vibration sensor disposed within a housing mounted on the exterior of the commode;
generating a processed vibration signal at a signal shaping circuit;
determining a urinary episode characteristic at a controller comprising a trained classifier coupled to the sensor receiving the processed vibration signal; and
generating an output corresponding to the urinary episode characteristic.

12. The method of claim 11 wherein determining the urinary episode characteristic comprises determining at least one of a urinary frequency, urinary flow strength and duration, a number of episodes within a urination episode, a low strength and duration of sub-episodes within the episode.

13. The method of claim 11 wherein generating the vibration signal comprises generating the vibration signal from the vibration sensor disposed in a commode wall.

14. The method of claim 11 wherein generating the vibration signal comprises generating the vibration signal from a piezoelectric sensor.

15. The method of claim 14 wherein generating the vibration signal comprises generating the vibration signal from a piezoelectric sensor comprising a cantilever comprising a first end fixed within the housing at a base, said cantilever comprising a second end comprising a ballast, said sensor comprising a piezoelectric layer disposed between the ballast and the base.

16. The method of claim 11 further comprising amplifying and denoising the vibration signal to formed the processed vibration signal.

17. The method of claim 16 further comprising band pass filtering the vibration signal to form the processed vibration signal.

18. The method of claim 16 further comprising forming a denoised and demodulated signal and identifying a urinating individual based on the denoised and demodulated signal.

19. The method of claim 18 wherein the generating a flap leak signal based on the denoised and demodulated signal.

20. The method of claim 16 further comprising analyzing urinary episodes and sub-episodes based on energy therein.

21. A urination monitoring system for a commode comprising:
a housing coupled to an exterior of the commode;
a vibration sensor disposed within the housing generating a vibration signal corresponding to vibration of the exterior of the commode;
a signal shaping circuit coupled to the vibration sensor generating a processed vibration signal;
a controller comprising a trained classifier coupled to the sensor receiving the processed vibration signal;
an analyzer determining a urinary episode characteristic from said urinary episode comprising at least one of a urinary frequency, urinary flow strength and duration, a number of episodes within a urination episode, a low strength and duration of sub-episodes within the episode; and
a display coupled to the analyzer generating screen display comprises a data area displaying data corresponding to the urinary episode characteristic.
